(19) United States
(12) Patent Application Publication
(10) Pub. No.: US 2025/0354125 A1
(43) Pub. Date: Nov. 20, 2025

(54) HIGH-THROUGHPUT LONG-TERM CULTURED ENDOTHELIAL ORGANOID WITH ANGIOGENESIS

(71) Applicant: Georgia Tech Research Corporation, Atlanta, GA (US)

(72) Inventors: Shuichi Takayama, Atlanta, GA (US); Alejandro De Janon, Atlanta, GA (US); Jeeyoung Kim, Seoul (KR); Jihoon Lee, Atlanta, GA (US)

(21) Appl. No.: 19/209,241

(22) Filed: May 15, 2025

Related U.S. Application Data
(60) Provisional application No. 63/649,046, filed on May 17, 2024.
(60) Provisional application No. 63/649,612, filed on May 20, 2024.

(51) Int. Cl. C12N 5/071; C12N 5/077; G01N 33/50

(52) U.S. Cl. CPC C12N 5/069; C12N 5/0652; C12N 5/0697; G01N 33/5064; G01N 33/5088; C12N 2502/08; C12N 2502/13; C12N 2513/00; C12N 2527/00; G01N 2500/10

(57) ABSTRACT

In accordance with at least one aspect of this disclosure, an in vitro cell construct includes, a cellular layer including endothelial cells having undergone a biological transformation defining a three-dimensional structure with vasculature formed around basement membrane mimetic gel inner core.

### CROSS-REFERENCE TO RELATED APPLICATIONS

This application claims priority to and the benefit of U.S. Provisional Application Nos. 63/649,046, filed May 17, 2024 and 63/649,612, filed May 20, 2024, the entire contents of each application is herein incorporated by reference in its entirety.

### TECHNICAL FIELD

The present disclosure relates to organoids and more particularly to high-throughput long-term cultured endothelial organoid with angiogenesis.

### BACKGROUND

HUVECs (Human Umbilical Vein Endothelial Cells) are primary cells isolated from the vein of umbilical cord and widely used in research to study various aspects of endothelial cell biology, including hypoxia/normoxia, immune response, angiogenesis, coagulation and inflammation. Endothelial cell is most related to vascular, so there are many works using co-culture system to study for angiogenesis, inflammation vascular related to diseases. In published works, there are many 2D HUVECs and spheroid related research, but there are no HUVEC only organoid culture published. Specially for angiogenesis assay, most used method is short-term (˜1 day) HUVEC/Matrigel tube or angiogenesis assay using hydrogel-filled microfluidic channels in co-culture with fibroblasts to mimic vessel structure.

Organoid is widely used for organ mimic model in several decades. The organoid, generally with higher cell heterogeneity, has structural and functional similarities to actual organs and more relevant to actual human organ system. An organoid model with excellent biocompatibility was implemented in vitro for use in disease research and drug development. Despite rapid advances in the field of organoid technology in the past decade, the lack of functional vasculature has been widely regarded as one of the major barriers hindering the effective recapitulation of in vivo physiology. To date, fully functional intravascular perfusion of organoids has only been reported through transplantation, which negates many of the perceived advantages of using organoids as a convenient in vitro model of organ function and disease.

The conventional techniques have been considered satisfactory for their intended purpose. However, there is an ever-present need for improved systems and methods for engineered in vitro platforms incorporating functional vasculature throughout the embedded organoids to broaden the biomedical applications of organoid technology. This disclosure provides a solution for this need.

### SUMMARY

In accordance with at least one aspect of this disclosure, an in vitro cell construct includes, a cellular layer including endothelial cells having undergone a biological transformation defining a three-dimensional structure with vasculature formed around basement membrane mimetic gel inner core.

The cellular layer can be a monolayer comprising only endothelial cells and the monolayer can be the only cellular layer surrounding the inner core. In certain embodiments, the inner core can include a hydrogel core formed of one or more of: Matrigel or Matrigel in combination with one or more of: collagens, fibronectin, vitronectin, hyaluronan, proteoglycan, glycoprotein, decellularized extracellular matrix, fibrin, fibrinogen, and/or DNA, RNA, and other nucleic acids.

In certain embodiments, the cellular layer can be an exterior cellular layer surrounding the inner core, where the endothelial cells of the exterior cellular layer are configured to form the vasculature of the three-dimensional structure via invaginating from the exterior cellular layer into the inner core.

In certain embodiments, the biological transformation can be or include endothelial to mesenchymal cell transformation (EndMT) whereby the endothelial cells of the cellular layer transition to mesenchymal-type cells exhibiting structural properties of mesenchymal cells. In certain embodiments, tension exerted on the stretched endothelial cells of the cellular layer, e.g., biological factors and culture conditions, induces the EndMT and generation of extracellular matrix, thereby stabilizing the three-dimensional structure.

In certain embodiments, the three-dimensional structure can be a spheroid, and the cell construct can be an organoid with organized endothelium and cellular heterogeneity. In certain embodiments, the three-dimensional structure can form in three hours or less from introduction of the endothelial cells to the basement membrane mimetic gel. In certain embodiments, the three-dimensional structure can form in two hours or less from introduction of the endothelial cells to the basement membrane mimetic gel. In certain embodiments, the three-dimensional structure can form in one hour or less from introduction of the endothelial cells to the basement membrane mimetic gel The three-dimensional structure can remain stable for more than 60 days. In certain embodiments, the three-dimensional structure can remain stable for more than 65 days. In certain embodiments, the three-dimensional structure can remain stable for more than 70 days. In certain embodiments, the three-dimensional structure can remain stable for 78 days.

In certain embodiments, the extracellular matrix produced by the EndMT can be endothelium-supportive extracellular matrix configured to support the stability of three-dimensional structure for an extended culture (e.g., more than 60 days) thereby providing culture conditions to allow the endothelial cells of the extracellular layer to undergo EndMT reversion. In certain embodiments, the extended culture can be 78 days, where at 78 days, EndMT reversion is induced or promoted.

In certain embodiments, the endothelial cells can be derived from one or more of: human umbilical vein endothelial cells (HUVECs), cardiac endothelial cells, dermal endothelial cells, brain endothelial cells, cancer endothelial cells, retinal endothelial cells, arterial endothelial cell, placental endothelial cell, liver endothelial cell, kidney endothelial cell, bone marrow endothelial cells, liver sinusoidal endothelial cells, lymphatic endothelial cells, glomerular endothelial cells, placental endothelial cells, brain endothelial cells, retinal endothelial cells, cancer endothelial cells.

In certain embodiments, the cellular layer can be a first cellular layer comprising endothelial cells, and the construct can further include a second cellular layer comprising mesenchymal or stromal cells co-cultured with the endothelial cells. In certain such embodiments, based on a first culture condition, the first cellular layer and second cellular layer can self-assemble into the three-dimensional structure around the core, such that the mesenchymal or stromal cells of the second cellular layer are outward of the core, and the endothelial cells of the first cellular layer are outward of mesenchymal or stromal cells of the second cellular layer. Alternatively, based on a second culture condition, the endothelial cells remain within three-dimensional structure to form a vascular network but not form a core aggregate. The first culture condition can include introducing the cells to a seeding media having 1-10% (vol/vol) or more fetal bovine serum and/or wound healing and/or cell migration-inducing growth factor. The second culture condition can include introducing the cells to a seeding media having 5 ng/ml of vascular endothelial growth factor (VEGF) and 5 ng/ml of fibroblast growth factor (FGF). In certain embodiments, the mesenchymal or stromal cells can be derived from one or more of: fibroblasts, smooth muscle cells, adipocytes and preadipocytes, pericytes, osteoblasts.

In certain embodiments, one or more additional cell types can be incorporated into the cellular layer, the one or more additional cell types including but not limited to: cancer cells, leukocytes, leukocyte progenitor or stem cells, and/or hematopoietic progenitor or stem cells, immune cells.

In certain embodiments, the construct can be an organoid configured for use in diagnostics, cell production, pharmaceutical testing, toxicology, cell engineering, EndMT reversion study, or the like.

In accordance with at least one aspect of this disclosure, a method of producing an in vitro cell construct can include, introducing a selection of endothelial cells to a seeding media having a basement membrane mimetic gel for allowing the endothelial cells to encapsulate at least a portion of basement membrane mimetic as an exterior cellular layer, the seeding media further including on or more of fetal bovine serum and/or wound healing and/or cell migration-promoting growth factors to form a seeding mixture, and centrifuging the seeding mixture to promote the endothelial cells to mechanically stretch around the portion of the basement membrane mimetic gel to form the exterior cellular layer thereby generating a three-dimensional structure where the exterior cellular layer is under tension.

In certain embodiments, only endothelial are introduced to the seeding media, such that the only exterior cellular layer formed around the core includes only endothelial cells as a monolayer, and wherein the tension generated by the mechanical stretch of the endothelial cells induces a biological transformation of the endothelial cells. In certain embodiments, the biological transformation is EndMT. In certain embodiments, the method can include, culturing the endothelial cells for an extended culture period (e.g., greater than 60 days) allows for EndMT reversion of the endothelial cells.

In certain embodiments, the method can further include, introducing a selection of mesenchymal or stromal cells to the seeding media such that the seeding mixture includes an endothelial cell and mesenchymal or stromal cell co-culture. In certain embodiments, the seeding mixture can include VEGF and FGF, where in such embodiments, the concentration of both pro-angiogenic factors causes the endothelial cells to form a second exterior cellular layer outside of the mesenchymal cell exterior cellular layer. In certain embodiments, wherein the seeding mixture does not include pro-angiogenic factors thereby causing the mesenchymal cells to remain within three-dimensional structure while the endothelial cells localize at an interior of the construct to form an aggregate at the core of the three-dimensional structure.

In certain embodiments, the pro-angiogenic factors include one or more of: fetal bovine serum and/or wound healing and/or cell migration-inducing growth factor, where the concentration of the pro-angiogenic factors can be about 1% to about 10% volume concentration (vol/vol). In certain embodiments, the pro-angiogenic factors can include one or more of: vascular endothelial growth factor (VEGF) and/or fibroblast growth factor (FGF), where the concentration of the pro-angiogenic factors can be about 5 ng/ml.

In accordance with at least one aspect of this disclosure, a method, can include, performing one or more of diagnostics, cell production, pharmaceutical testing, toxicology, cell engineering, EndMT reversion study, or the like on the in vitro cell construct shown and described herein.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 is a schematic diagram showing an endothelial organoid culture in accordance with an exemplary embodiment of this disclosure, where the left-hand figure shows the organoid after centrifuge and the right-hand figure show the organoid culture are less than one day of culturing;

FIG. 2 is a schematic diagram showing sprouting of an organoid, e.g., the exemplary endothelial organoid of FIG. 1;

FIG. 3 is a series of bright field images showing the progression of organoid formation in culture from 0 hours to 24 hours;

FIG. 4 shows plots identifying the cross-sectional area and circularity of cultured organoids;

FIG. 5 shows images of hematoxylin and eosin (H&E) stained organoid sections taken at day three of culture and day seven of culture;

FIG. 6 shows an immunofluorescence analysis of an exemplary organoid at 28 days of culture and 48 days of culture, and shown in a single plane and in a merged plane;

FIG. 7 is a series of bright field images of a cultured sprouted organoid taken at days 3, 7, 14, 28, 51, and 61, where FIG. 7a is a magnification of the bright field image of the day 61 culture shown in FIG. 7, showing the developed vasculature;

FIG. 8 is a confocal immunofluorescence analysis of an exemplary P4 organoid shown in a single plane after 6 and 18 days of culture, and in a merged plane after 6, 18, 26, and 43 days of culture;

FIG. 9a is an immunofluorescence analysis of an exemplary integrin α6 stained P4 organoid, showing signals observed both on the surface of the organoid and internal to the organoid;

FIG. 9b is an immunofluorescence analysis of an exemplary CD34 stained organoid, showing progenitor cell signals observed at day 3 and day 10 of culture;

FIGS. 10a-10e are immunofluorescence analysis of exemplary actin stained organoids at various days of culture, showing formation or lack of formation of structures inside the organoid;

FIG. 11 shows bright field images of stimulation endothelial organoid before treatment with LPS and TNF-α and 24 hours after treatment;

FIGS. 12a-12d single-cell transcriptomics data is shown, wherein:

FIG. 12a is a UMAP visualization showing distinct cellular clusters that comprise the system;

FIG. 12b is a pseudotime trajectory analysis illustrating the dynamic progression and connectivity between clusters;

FIG. 12c shows expression of canonical marker genes used to annotate cluster identities; and

FIG. 12d is a Gene Ontology (GO) enrichment analysis showing the top five enriched GO terms for each cluster;

FIGS. 13a-14b show a endothelial-to-mesenchymal transition (EndMT) analysis, wherein FIG. 13a shows expression of transcription factors and markers associated with EndMT;

FIG. 13b shows immunofluorescence staining of fibronectin in organoids;

FIG. 13c shows immunofluorescence staining of alpha smooth muscle actin (αSMA) in organoids;

FIG. 14a shows an organoid stimulation with IL-1β and TGF-β, and treatment using the PIEZO1 inhibitor GsMTx4; and

FIG. 14b shows organoid stimulation with treatment with ROCK inhibitor Y-27632;

FIGS. 15a-15c show Cell-Cell interactions maps, wherein:

FIG. 15a shows Cell-cell communication networks predicted by CellChat2 analysis;

FIG. 15b shows an extracellular matrix (ECM) production network focused on collagen and fibronectin; and

FIG. 15c shows an analysis of angiogenic and sprouting signaling pathways;

FIG. 16 is a schematic diagram of an embodiment of a Vascular Mesenchymal Organoids (VMO) formation process in accordance with at least one aspect of this disclosure, the formation process combining mesenchymal stem/stromal cells (MSCs), endothelial cells, and a basement membrane mimetic gel;

FIG. 17 shows time-lapse images showing organoid formation during the first 3 days across different EC:MSC ratios;

FIG. 18 shows an immunofluorescence analysis of organoids at different seeding ratios;

FIG. 19 shows a quantification of the cross-sectional area of organoids at different seeding ratios;

FIG. 20 shows circularity measurements of organoids over 60 days of culture;

FIG. 21 shows data visualizing VMOs proliferation, vascularization ECM assembly, and inflammation as compared to spheroids;

FIG. 22 shows time-lapse bright field images of organoids formed from different HUVEC:MSC ratios over three days of culture;

FIG. 23, shows immunofluorescence analysis of MSC (CD90) and HUVEC (VE-CADHERIN) distribution within the organoids at different seeding ratios after 14 days of culture;

FIG. 24, shows immunofluorescence analysis of organoid core structure on culture days 3, 14, and 60;

FIG. 25 shows organoid culture kinetics at different seeding ratios, comparing co-cultured organoids to HUVEC-only organoids;

FIG. 26a plots cross-sectional area of organoids with different seeding ratios over eight weeks of culture;

FIG. 26b plots organoid circularity of organoids with different seeding ratios over eight weeks of culture;

FIG. 27a is an immunofluorescence analysis of a cross-section of an exemplary organoid showing a microenvironment of the organoid;

FIG. 27b is a 3D reconstruction of the immunofluorescence analysis of the exemplary organoid of FIG. 27a;

FIG. 28a, is an immunofluorescence analysis of the production of extracellular matrix (ECM) molecules in situ, including fibronectin, collagen I, and laminin V;

FIG. 28b shows an immunofluorescence analysis of mural cells are visible on the exemplary organoid's surface;

FIGS. 29 and 30 show immunofluorescence analysis of an exemplary organoid staining with alpha-SMA (smooth muscle actin), wherein:

FIG. 29 shows a general distribution of vascular smooth muscle cells; and

FIG. 30 is an enlarged view of the distribution shown in FIG. 29;

FIGS. 31-33 show immunofluorescence analysis of vasculature formation in exemplary Co-Culture organoids vs exemplary HUVEC Mono-Culture organoids, wherein:

FIG. 31 shows the location of HUVECs (VE-CADHERIN) and endothelial progenitors (CD34) after three days of culture;

FIG. 32 shows location of the vasculature in HUVEC mono-culture versus co-culture after 14 days of culture;

FIG. 33 shows location of the vasculature in HUVEC mono-culture versus co-culture after 60 days of culture;

FIGS. 34-36 show 3-D reconstructions of the vasculature formation in Co-Culture vs HUVEC Mono-Culture shown in FIGS. 31-33, wherein:

FIG. 34 is a 3D reconstruction of FIG. 31;

FIG. 35 is a 3D reconstruction of FIG. 32; and

FIG. 36 is a 3D reconstruction of FIG. 33;

FIGS. 37-43 show data relating to osteogenesis in 50:50 organoids cultured in osteogenic versus traditional growth media, wherein:

FIG. 37 is an image showing organoids cultured for 3 weeks in osteogenic growth medium and stained with Alizarin Red to identify calcium nodules, pointing out calcium nodules with arrows;

FIG. 38 is an image showing organoids cultured for 3 weeks in traditional growth media and stained with Alizarin Red to identify calcium nodules,

FIG. 39 shows immunofluorescence analysis on organoids subjected to osteogenesis for 4 weeks;

FIG. 40 shows qPCR analysis of RUNX2 gene expression performed after 14 days in growth medium;

FIG. 41 shows qPCR analysis of RUNX2 gene expression performed after 28 days in growth medium

FIG. 42 shows qPCR analysis of OPN gene expression performed after 14 days in growth medium;

FIG. 43 shows qPCR analysis of OPN gene expression performed after 28 days in growth medium; and

FIG. 44 shows bulk RNAseq though day 78 of culture showing the increase/decrease in EndMT and endothelial markers.

### DETAILED DESCRIPTION

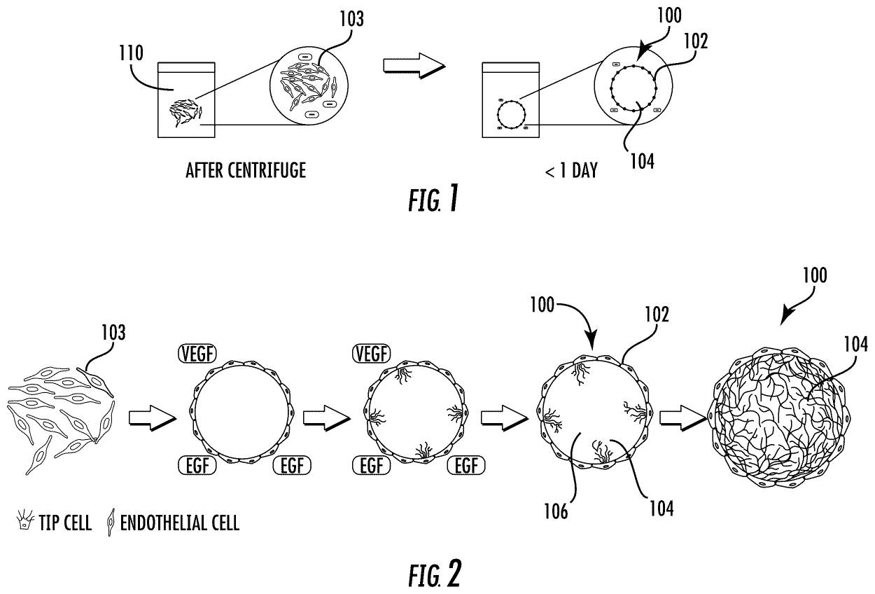

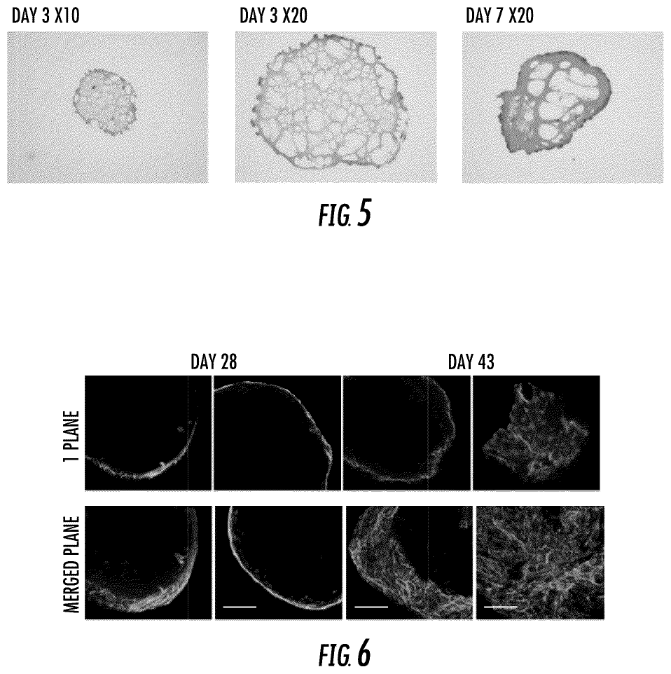

Reference will now be made to the drawings wherein like reference numerals identify similar structural features or aspects of the subject disclosure. For purposes of explanation and illustration, and not limitation, an illustrative view of an embodiment of an in vitro cell construct in accordance with the disclosure is shown in FIG. 1 and is designated generally by reference character 100. Other embodiments and/or aspects of this disclosure are shown in FIGS. 2-17.

Human Umbilical Vein Endothelial Cells (HUVECs) are primary cells isolated from the umbilical cord vein, as valuable model for studying endothelial cell function. This disclosure introduces a novel, long-lived and the first endothelial only organoid model in the world. As will be discussed further herein, HUVEC organoids were cultured in commercial 384-well ultra-low attachment (ULA) microplates. Basic characterization of the organoids was performed image analysis, assessing size and circularity of organoid for day 67. Notably, the novel organoids as described herein developed angiogenesis internally, confirmed by actin staining structures resembling vessels.

From Bulk RNAsequencing data, the inventors confirmed increase in genes related to angiogenesis, including the CD34, known for tip cell marker and as endothelial progenitor cell marker do crucial role in angiogenesis. Inflammation analysis performed Lipopolysaccharides (LPS) and Tumor Necrosis Factor-alpha (TNF-alpha) stimulation showed E-selectin signals on the organoids surface. These are the potential of the long-lived endothelial only organoids as a novel model for studying various aspects of endothelial cell function, including inflammation, vascularization and response to infection. There are multiple diseases known to accompany loss of pericytes. In vitro cultures of endothelial cells without support cells such as fibroblasts, MSCs or other pericyte or mural cells, however, are known to be short lived. The need for models to study vasculature lacking pericytes conflicts with the requirement of in vitro endothelial cell cultures to have exogeously-added pericytes or pericyte-like cells for culture stability lead to a gap in in vitro models to study diseases with vascular dysfunction that have loss of the pericytes. Accordingly, the invention described herein includes HUVEC organoid 3D models that do not require pericytes for long-term (at least 78 days) culture and analysis.

In accordance with at least one aspect of this disclosure, as shown in FIGS. 1-3, an in vitro cell construct 100 includes, a cellular layer 102 including endothelial cells 103 having undergone a biological transformation defining a three-dimensional structure with vasculature 104 formed around basement membrane mimetic gel inner core 106.

The cellular layer 102 can be a monolayer comprising only endothelial cells 103 (e.g., as shown and described with respect to FIGS. 1-15c) and the monolayer can be the only cellular layer 102 surrounding the inner core 106. In certain embodiments, the inner core 106 can include a hydrogel core of one or more of: Matrigel or Matrigel in combination with one or more of: collagens, fibronectin, vitronectin, hyaluronan, proteoglycan, glycoprotein, decellularized extracellular matrix, fibrin, fibrinogen, and/or DNA, RNA, and other nucleic acids.

In certain embodiments, the cellular layer 102 can be an exterior cellular layer surrounding the inner core 106 and the endothelial cells 103 of the exterior cellular layer can be configured to form the vasculature 104 of the three-dimensional structure via invaginating from the exterior cellular layer 102 into the inner core 106 as shown in FIG. 2 for example.

In certain embodiments, the biological transformation can be or include endothelial to mesenchymal cell transformation (EndMT) whereby the endothelial cells 103 of the cellular layer 102 transition to mesenchymal-type cells exhibiting structural properties of mesenchymal cells. In certain embodiments, tension exerted on the stretched endothelial cells of the cellular layer, e.g., biological factors and culture conditions, induces the EndMT and generation of extracellular matrix, thereby stabilizing the three-dimensional structure.

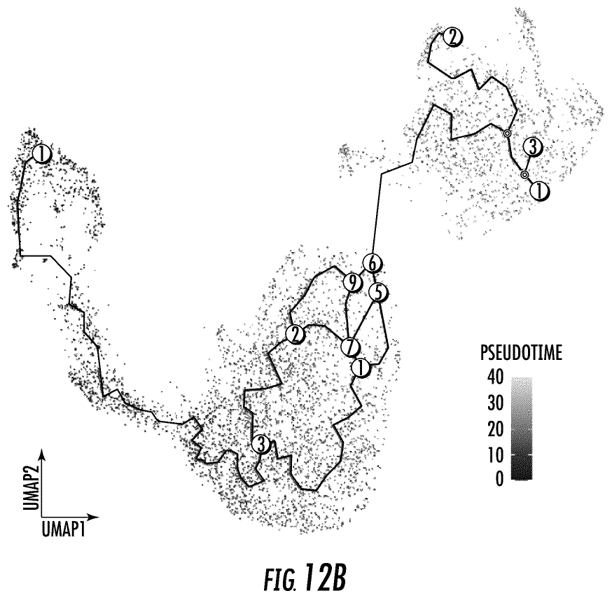

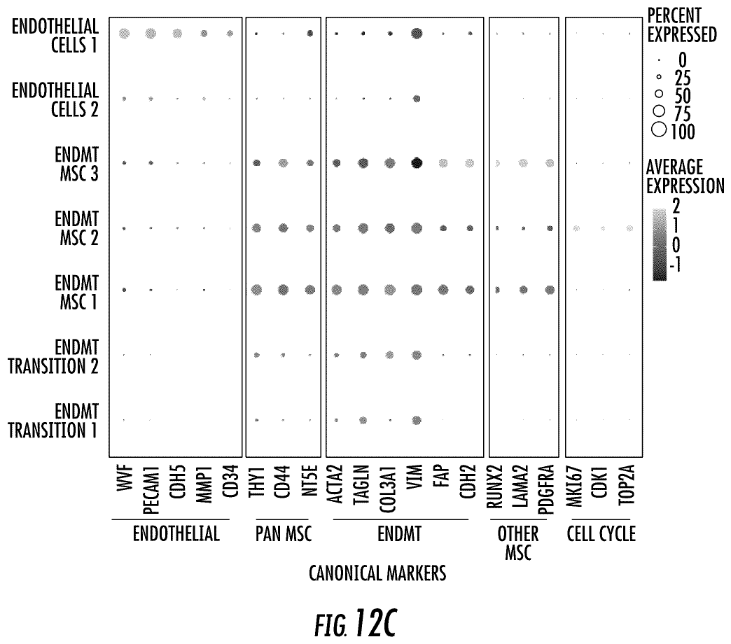

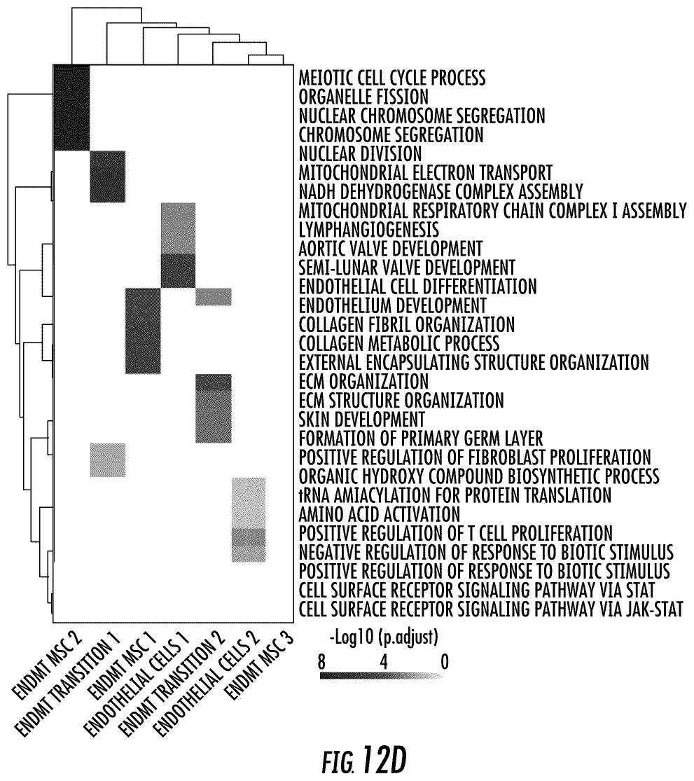

In certain embodiments, the three-dimensional structure can be a spheroid, and the cell construct can be an organoid with organized endothelium and cellular heterogeneity. In certain embodiments, the three-dimensional structure can form in three hours or less from introduction of the endothelial cells to the basement membrane mimetic gel. In certain embodiments, the three-dimensional structure can form in two hours or less from introduction of the endothelial cells to the basement membrane mimetic gel. FIGS. 12a-15c detail the EndMT.

FIGS. 12a-12d show single-cell transcriptomics. FIG. 12a is a UMAP visualization showing distinct cellular clusters that comprise the system, including two endothelial clusters, two Endothelial-to-Mesenchymal Transition (EndMT) transition clusters, and three EndMT-derived mesenchymal stem cell (MSC) clusters. FIG. 12b shows a pseudotime trajectory analysis illustrating the dynamic progression and connectivity between clusters, with the trajectory initiating in endothelial clusters and terminating in EndMT clusters, suggesting that these clusters are derived from endothelial cells. FIG. 12c shows expression of canonical marker genes used to annotate cluster identities: endothelial cells are identified by high expression of PECAM1, CDH5, and VWF; mesenchymal stem cells by THY1 and CD44; EndMT cells by ACTA2, TAGLN, and CDH2; and proliferating cells by the cell cycle markers MKI67 and TOP2A. FIG. 12d shows a Gene Ontology (GO) enrichment analysis showing the top five enriched GO terms for each cluster, highlighting key biological processes that characterize and distinguish the identified cell populations.

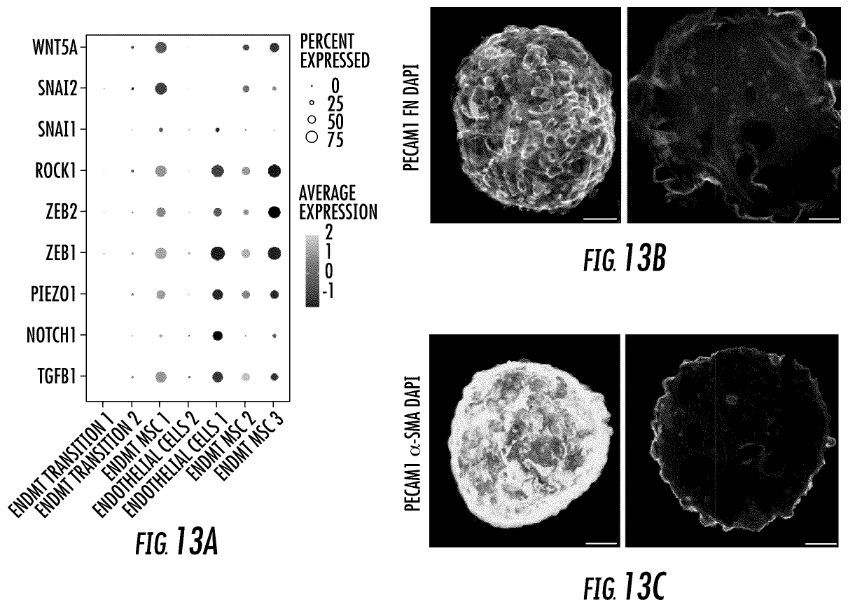

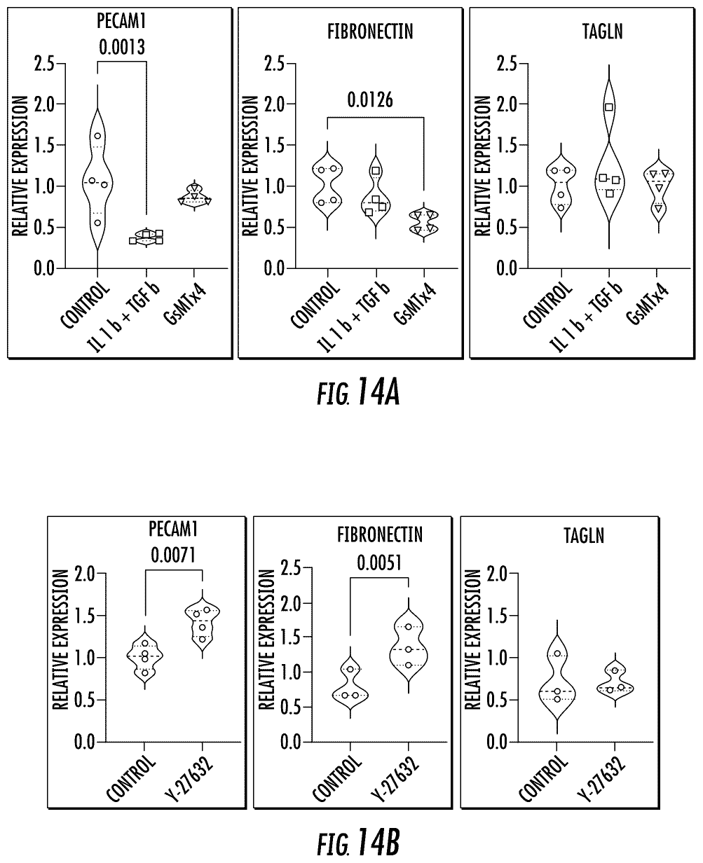

FIGS. 13a-14b show an endothelial-to-mesenchymal transition analysis. In FIG. 13a, expression of transcription factors and markers associated with Endothelial-to-Mesenchymal Transition (EndMT) is shown, highlighting the enrichment of ROCK1 and PIEZO1 in endothelial cell clusters. FIG. 13b shows immunofluorescence staining of fibronectin in organoids shows its distribution as an EndMT marker and FIG. 13c shows immunofluorescence staining of alpha smooth muscle actin (αSMA), another EndMT marker. The scale bars in FIGS. 13b and 13c represent 50 m.

FIG. 14a shows an organoid stimulation with IL-1β and TGF-β, along with treatment using the PIEZO1 inhibitor GsMTx4, revealed that IL-1β reduced PECAM1 expression-indicating an inflammatory response-without affecting fibronectin or TAGLN levels. In contrast, GsMTx4 decreased fibronectin expression but did not alter TAGLN, suggesting partial EndMT reversion. FIG. 14b shows treatment with the ROCK inhibitor Y-27632 increased PECAM1 expression, suggesting partial rescue of endothelial identity, but also elevated fibronectin levels; TAGLN expression remained unchanged.

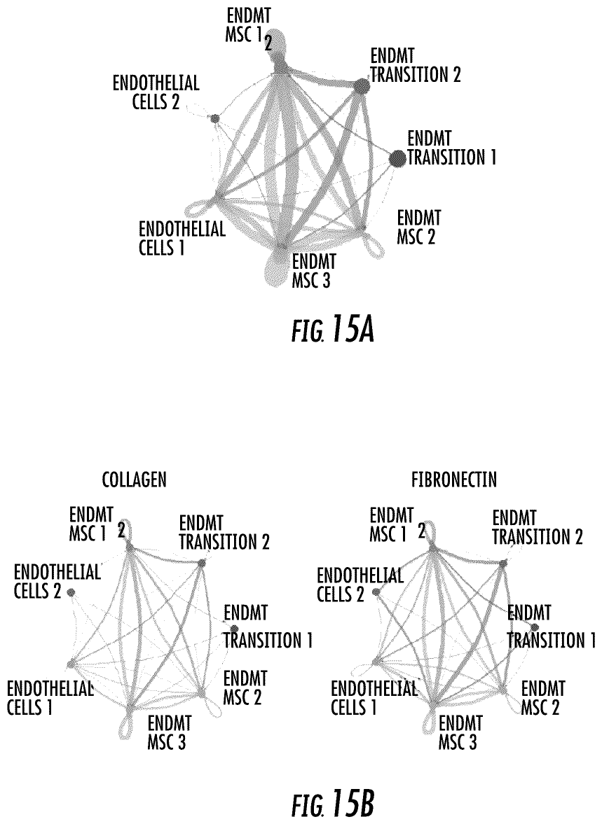

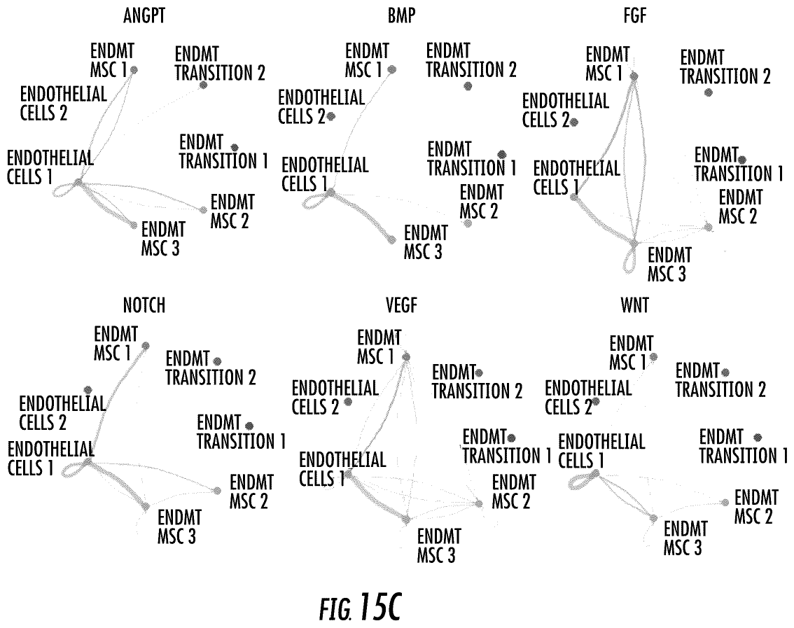

FIGS. 15a-15c show Cell-Cell interactions maps. In FIG. 15a, Cell-cell communication networks predicted by CellChat2 analysis is shown, illustrating interactions between different cell populations within the system. FIG. 15b shows extracellular matrix (ECM) production network focused on collagen and fibronectin, showing the majority of ECM components are produced by EndMT-derived MSC populations. In FIG. 15c, an analysis of angiogenic and sprouting signaling pathways is shown, revealing that most angiogenic factors are produced by endothelial cells, with supportive signals originating from EndMT-derived MSCs.

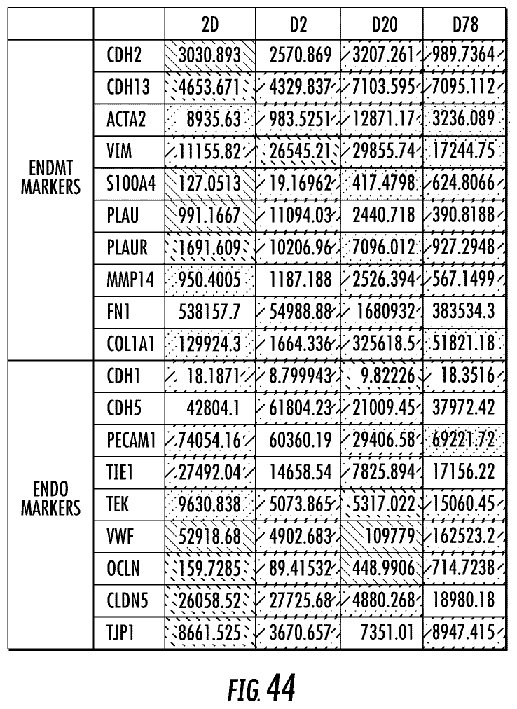

In certain embodiments, the extracellular matrix produced by the EndMT can be endothelium-supportive extracellular matrix configured to support the stability of three-dimensional structure for an extended culture (e.g., more than 60 days) thereby providing culture conditions to allow the endothelial cells of the extracellular layer to undergo EndMT reversion. In certain embodiments, the extended culture can be 78 days, where at 78 days, EndMT reversion is induced or promoted. This can be visualized in FIG. 44, for example.

As shown in FIG. 44, bulk RNAseq data values for EndMT markers and Endothelial markers are provided. This data shows that EndMT markers peak at Day 20 then reverse at Day 78. The long-term culture and eventual replacement of original Matrigel with cell produced matrix as well as reduction in endothelial tension and other biochemical factor changes allow for EndMT reversal. Note also the endothelial cell markers that show the opposite trend to EndMT where Day 20 shows reduction in Endothelial cell markers but then it rebounds by Day 78.

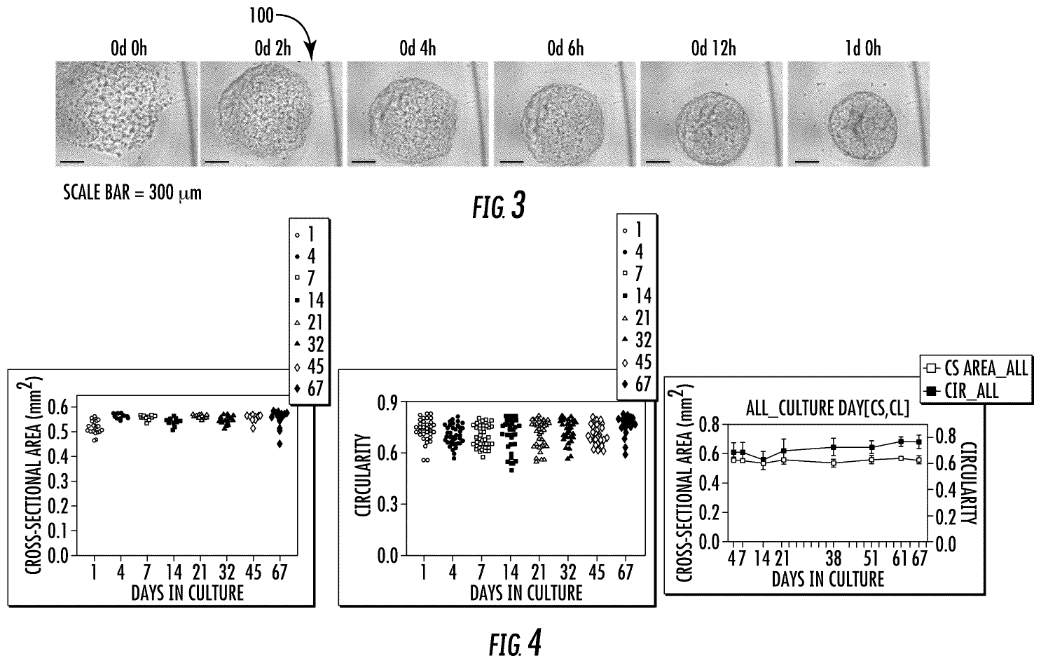

As shown in FIGS. 1-11, the three-dimensional structure can be a spheroid, and the cell construct can be an organoid. In certain embodiments, as shown in FIG. 3, the three-dimensional structure can form in three hours or less from introduction of the endothelial cells to the basement membrane mimetic gel. In certain embodiments, the three-dimensional structure can form in two hours or less from introduction of the endothelial cells to the basement membrane mimetic gel. In certain embodiments, the three-dimensional structure can form in one hour or less from introduction of the endothelial cells to the basement membrane mimetic gel/The three-dimensional structure can remain stable for more than 60 days, for example as shown in FIGS. 4-7. In certain embodiments, the three-dimensional structure can remain stable for more than 65 days. In certain embodiments, the three-dimensional structure can remain stable for more than 70 days.

In certain embodiments, the endothelial cells can be derived from one or more of: human umbilical vein endothelial cells (HUVECs), cardiac endothelial cells, dermal endothelial cells, brain endothelial cells, cancer endothelial cells, retinal endothelial cells, arterial endothelial cell, placental endothelial cell, liver endothelial cell, kidney endothelial cell, bone marrow endothelial cells, liver sinusoidal endothelial cells, lymphatic endothelial cells, glomerular endothelial cells, placental endothelial cells, brain endothelial cells, retinal endothelial cells, cancer endothelial cells.

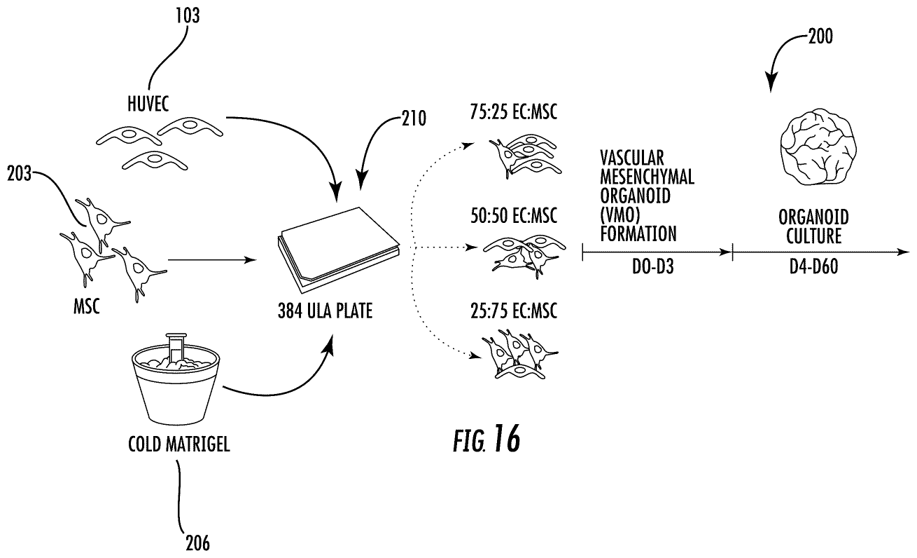

With reference now to FIGS. 16-24, in certain embodiments, the cellular layer 102 can be a first cellular layer 202 comprising endothelial cells, and the construct 200 can further include a second cellular layer 208 comprising mesenchymal or stromal cells co-cultured with the endothelial cells. Based on the particular culture condition, e.g., whether or not the culture includes pro-angiogenic factors, the first and second cellular layers may organize differently. With pro-angiogenic factors present, the first cellular layer 202 and second cellular 208 layer can self-assemble into the three-dimensional structure around the core 206, such that the mesenchymal or stromal cells of the second cellular layer 208 are outward of the core 206, and the endothelial cells of the first cellular layer 202 are outward of mesenchymal or stromal cells of the second cellular layer 208 as shown in FIG. 18. In culture conditions where pro-angiogenic factors are absent, the mesenchymal cells remain within three-dimensional structure while the endothelial cells localize at an interior of the construct to form an aggregate at the core of the three-dimensional structure. In certain embodiments, the mesenchymal or stromal cells can be derived from one or more of: fibroblasts, smooth muscle cells, adipocytes and preadipocytes, pericytes, osteoblasts.

In certain embodiments, one or more additional cell types can be incorporated into the cellular layer, the one or more additional cell types including but not limited to: cancer cells, leukocyte progenitor or stem cells, and/or hematopoietic progenitor or stem cells, or immune cells,

In certain embodiments, the construct can be an organoid configured for use in diagnostics, cell production, cell engineering, pharmaceutical testing, and/or in academic study such as studying EndMT reversion of the endothelial cells.

In accordance with at least one aspect of this disclosure, referring again to FIG. 1, a method of producing an in vitro cell construct can include, introducing a selection of endothelial cells 102 to a seeding media having a basement membrane mimetic gel and, in certain embodiments, pro-angiogenic factors or growth factors, in a seeding mixture 110. The amount of basement membrane mimetic gel can be just sufficient to allow for the encapsulation by the endothelial cells. The method further includes centrifuging the seeding mixture 110 to promote the endothelial cells 103 to mechanically stretch around the portion of the basement membrane mimetic gel to form the exterior cellular layer 102 thereby generating a three-dimensional structure where the exterior cellular layer is under tension.

In certain embodiments, such as shown in FIG. 1, only endothelial are introduced to the seeding media, such that the only exterior cellular layer formed around the core includes only endothelial cells. In certain embodiments, the biological transformation can be or include EndMT.

In certain embodiments, such as shown in FIG. 16, the method can further include, introducing a selection of mesenchymal or stromal cells 203 to the seeding media such that the seeding mixture 210 includes an endothelial cell and mesenchymal or stromal cell co-culture. In certain embodiments, the method can further include, introducing a selection of mesenchymal or stromal cells to the seeding media such that the seeding mixture includes an endothelial cell and mesenchymal or stromal cell co-culture. In certain embodiments, the seeding mixture can include pro-angiogenic factors VEGF and FGF, where in such embodiments, the concentration of both pro-angiogenic factors causes the endothelial cells to form a second exterior cellular layer outside of the mesenchymal cell exterior cellular layer.

In certain embodiments, the pro-angiogenic factors include one or more of: fetal bovine serum and/or wound healing and/or cell migration-inducing growth factor, where the concentration of the pro-angiogenic factors can be about 1% to about 10% volume concentration (vol/vol). In certain embodiments, the pro-angiogenic factors can include one or more of: vascular endothelial growth factor (VEGF) and/or fibroblast growth factor (FGF), where the concentration of the pro-angiogenic factors can be about 5 ng/ml.

In certain embodiments, the seeding mixture does not include pro-angiogenic factors thereby causing the mesenchymal cells to remain within three-dimensional structure while the endothelial cells localize at an interior of the construct to form an aggregate at the core of the three-dimensional structure.

In accordance with at least one aspect of this disclosure, a method, can include, performing one or more of diagnostics, cell production, cell engineering, pharmaceutical testing, and/or EndMT reversion study on the in vitro cell construct shown and described herein.

Vasculature is a critical component of tissue architecture, performing essential functions such as nutrient exchange, waste removal, and facilitating cellular communication. Beyond its role in normal tissue homeostasis, the vasculature also plays a role in the progression of various diseases. Pathological alterations in vascular structure and function are implicated in conditions such as vascular leakage, endothelial dysfunction, and chronic inflammation. Vascular alterations are particularly important in understanding physiology and developing effective therapies against cancer metastasis.

Microchannel-based systems that use preformed channels to guide extracellular matrix (ECM) gel formation, endothelial cell and fibroblast seeding, and vessel formation is currently regarded as one of the most representative systems. Vascularized organoids are emerging as another technique that allows endothelial cells to interact dynamically with their surrounding environment to self-organize into interconnected vessels. This self-organizing process replicates an aspect of in vivo vascular remodeling.

Vascularized organoids, e.g., as described herein, seek to provide a practical and physiologically relevant system that can reflect how blood vessels form and function in tissues. Vasculature development involves supporting cells such as pericytes and mural cells that promote angiogenesis, stabilize vessels, and maintain vascular integrity, at least in part, through providing an appropriate extracellular matrix for the endothelial cells. In certain embodiments, co-culturing endothelial cells with supporting cells like pericytes, mural cells, and mesenchymal stem/stromal cells (MSCs), can creates a more functional model.

In embodiments utilizing co-culture, MSCs are particularly important in reconstructing the bone marrow stroma, where they regulate angiogenesis and provide structural support. Space-filled, MSC-endothelial co-cultures generally referred to as spheroids simulate aspects of the bone marrow microenvironment but generally fail to develop a long-lived vasculature. These spheroids, in certain embodiments, consist of aggregates of elongated endothelial cells and MSCs with or without hydrogel, but the vessel diameters are small and with few branching networks. At the same time, induced pluripotent stem cell (iPSC) vascular organoids form a vascularized tissue with supporting cells and ECM production, are becoming an important tool to study vascularized tissues. Their use can involve challenges such as taking several weeks to achieve tissue maturation, having heterogeneous maturation, or low functionalization.

One disease that involves the vasculature is breast cancer metastasis to the bone marrow. Replicating this bone marrow metastasis in vitro is challenging due to the complexity of the bone marrow microenvironment and the specific interactions between cancer cells, the vasculature, and stromal components. Furthermore, in vivo rodent models rarely show metastasis to the bone, orthotopic implantation is challenging requiring exogeneous implants 26 or difficult intracardiac or intracardial arterial injection that can lead to animal death, off-target invasion and poor cell delivery. Thus, a robust endothelial-MSC platform, such as described in certain embodiments disclosed herein, may benefit investigation of breast cancer-stromal interactions in a controlled, physiologically relevant setting.

A common challenge in traditional organoid cultures, compared to spheroids which generally form nice consistent structures in a one-well one-spheroid format, is standardization and single organoid readouts. Embodiments of the novel EC Organoids and VMOs described herein provide free-floating one-well one-organoid format structures that are relatively large in a 384 well plate format providing the potential for high-throughput single organoid-based studies.

Embodiments of the EC Organoid and EC-MSC VMO model complement traditional spheroids by providing a standardized model which can be easier to work with. Additionally, embodiments can better replicate the bone marrow vasculature and cellular interactions. Embodiments of the VMO platform supports long-term endothelium stability and MSC multipotency, while also mimicking the vascular microenvironment through enhanced ECM, metabolic plasticity, immune modulation, and the presence of perivascular cells. Embodiments of the model offers a promising platform for studying bone marrow metastasis and cancer progression, providing valuable insights into cell behavior, niche dynamics, and treatment responses, making it an invaluable tool for therapeutic research.

### EXPERIMENTAL

### Materials and Methods—Endothelial Only Organoids

### Endothelial Cell Culture (2D)

Human umbilical vein endothelial cells (HUVECs) (ATCC, PCS-100-013, Lot 70032759) were grown in Vascular cell basal medium (VCBM) supplemented with rh VEGF (5 ng/mL), rh EGF (5 ng/mL), rh FGF basic (5 ng/mL), rh IGF-1 (15 ng/mL), L-glutamine (10 mM), Heparin sulfate (0.75 Units/mL), Hydrocortisone (1 μg/mL), Ascorbic acid (50 μg/mL), 2% (vol/vol) fetal bovine serum (FBS) (ATCC, PCS-100-041) and 1% (vol/vol) pen/strep. The medium was replaced every 2 days and cells were maintained in a humidified CO₂ incubator at 37° C. and 5% CO₂. HUVECs were cultured in T75 and T150 flasks and passaged at 80% confluency. Primary HUVECs were used until passage number 2 to 4. All experiments were performed with lot 70032759.

### Endo Organoid Culture (3D)

HUVEC Endothelial organoids were seeded in 384-well U-bottom ULA plates (S-bio #MS-9384UZ, lot 20839306) and centrifuged at 1,000 rpm for 5 min (142 rcf, Thermo Scientific Sorvall ST16 centrifuge). Endothelial organoid seeding volume was 25 μL per well. The HUVEC organoid seeding solution consisted of 120,000 HUVEC cells/mL, 160 μg/mL Matrigel (Corning #356-231, 10.7 mg/mL), 10% FBS (GeminiBio #900-108) and 0.24% methylcellulose (Methocel, Sigma Aldrich #94378). A key procedure being to add cold Matrigel into warm media to induce gelling.

### Live Organoid Imaging and Image Analysis

HUVEC Endothelial organoids were imaged using an EVOS FL 2 Auto microscope (ThermoFisher) with a 4× and 10× magnification. Bright field images with 4× magnification were analyzed using Python image analysis algorithm. The algorithm was adapted from Lee S, #Chang J J, #Kang S-M, Parigoris E, Lee J-H, Huh Y S, Takayama S. “High-throughput formation and image-based analysis of basal-in mammary organoids in 384-well plates” Sci Rep. 12, 317, (2022), the entire content of which is incorporated by reference herein. The Python program retains the original functionality in characterizing the circularity and cross-sectional area of imaged organoids. An additional supporting Python script was created to calculate pixel size and image area for bright field images; the original MATLAB script was specific to different image format. Cross sectional area data gained from the image analysis program was then scaled by a factor 2.0 to account for scaling issue inherent to the Python script. Manual cross-sectional area calculations showed that cross sectional area data gained by Python was lower by a factor 2. This could most likely be attributed to the nuanced differences in how masking operates in Python and MATLAB.

### Endothelial Organoid Embedding, Histology and H&E Staining

HUVEC Endothelial organoids were collected from the 384-well U-bottom ULA Plate and washed with PBS. Organoids were fixed for 30 min with 4% Paraformaldehyde (PFA, Alfa Aesar) at room temperature and washed with PBS. Organoids were stained for 10 min in 0.5% methylene blue (RICCS Chemical Company, #4850-4) to aid in the visualization of organoids and washed with PBS to remove excess dye. A small amount of optimal cutting temperature (OCT, Tissue-Tek #62550-01) was added to cryomold. 10 to 15 organoids were transferred to cryomold and covered with OCT. Isopentane (Sigma #M32631) was cooled in liquid nitrogen, and samples were flash frozen and stored at −80° C. HUVEC organoid block was sectioned, and 10 μm thickness sections of the organoids were obtained using a CryoStar NX70 cryostat (Thermo Fisher) and put 2-3 sections on each slide. For the hematoxylin and eosin (H&E) staining, organoids section slides were brought to room temperature and stained with an ST5010 Autostainer XL (Leica). Slides were cover slipped with Xylene and Cytoseal 60 (Richard-Allan Scientific) and imaged in color with a DMi1 microscope (Leica) and 10× air objective.

### Immunofluorescence and Confocal Imaging

Fixed HUVEC Organoids were washed with PBS and permeabilized with 0.1% Tween in PBS for 10 min at 4° C. Samples were incubated with Organoid washing buffer (OWB, 0.2% BSA/0.1% Triton-X-100 in PBS) to prevent non-specific binding for 15 min at 4° C. Samples were incubated with primary antibodies in OWB at 4° C. overnight. Primary antibodies include: SELE Rabbit pAb (ABclonal, #A2191, Lot 5500024697, 1:1,000 dilution), Human VE-Cadherin Antibody (R&D systems, #AF938, 1:40 dilution) and Mouse anti-CD34 antibody (Clone QBEND/10, Invitrogen #MA1-10202, 1:500 dilution). Samples were washed with OWB and incubated with secondary antibodies or Alexa Fluor 647 Phalloidin (Invitrogen, #A22287, Lot 2170291, 1:400 dilution) in OWB for 2 hours at room temperature (RT). Secondary antibodies include: Goat anti-Rabbit Alexa Fluor 647 (Invitrogen, #A-21244, Lot 2134003, 1:500 dilution), Donkey anti-rat Alexa Fluor 488 (1:2,000 dilution), Goat anti-mouse Alexa Fluor 488 (Thermo Fisher, #A-11001, 1:2,000 dilution) and Donkey anti-mouse Alexa Fluor 568 (Invitrogen #A10037, 1:1,000 dilution). Organoids were washed with PBS and incubated with DAPI in PBS for 30 min at RT and imaged using Spinning disk Confocal microscope (Nikon, CSU-W1) with 10×, 20× and 60× magnification. E-selectin, Integrin α6 and CD34 stained organoids were performed without permeabilization step, VE-Cadherin and Phalloidin stained organoids were performed with permeabilization step

### Bulk RNA Sequencing

For bulk RNA sequencing, a total of seven experimental conditions, each replicated in triplicate, were prepared. These conditions comprised two groups: low (P4) and high (P16) passage HUVECs, both cultured in 2D, along with their corresponding organoids at two identical timepoints (Days 2 and 20), with an additional extended timepoint (Day 78) specifically for the P4 conditions. To extract cell lysates from organoids, five to ten organoids per replicate were harvested from ULA plates into DNase- and RNase-free tubes (Eppendorf, 022363344). Adherent cells from 2D cultures were collected using a cell scraper (Sarstedt, 83.1380). The pooled cells/organoids were then lysed in RLT lysis buffer (Qiagen, 79216) containing 1.0% beta-mercaptoethanol (Sigma-Aldrich, M3148). Subsequently, the lysates were transported on dry ice to Emory Integrated Genomics Core (EIGC), Emory University, for further processing and sequencing.

RNA isolation was performed using the Quick-RNA Microprep Kit (Zymo Research), followed by cDNA synthesis using the SMART-Seq® v4 Ultra® Low Input RNA Kit (Takara Bio). The final sequencing libraries were constructed using the Nextera XT kit (Illumina), with Nextera XT chemistry used to tagment 200 μg of DNA and add adapters in a single step. The fragmented and tagged DNA underwent 12 cycles of amplification to incorporate dual-indexing sequencing adapters, resulting in the final library. Quality control metrics included assessment of concentration and library size using a UV Qubit fluorometer for quantitation and a Bio/Fragment Analyzer trace for size distribution and concentration determination. Library scores were assigned based on these parameters, with only libraries meeting passing criteria proceeding to sequencing on a NovaSeq (Illumina) SP flowcell. The resulting read depth for the run was approximately 40 million reads per sample.

### Results and Discussion—Endothelial Only Organoids

### Endothelial Organoid Formation in 384-Well ULA Plate and Image Analysis

Commercial 384-well plate was used for culture HUVEC organoid. Organoid were seeded on ULA plate with various growth factors and matrigel, and centrifuged for 10 mins to help cells easy to aggregation. (See, e.g., Lee et al. “High-throughput formation and image-based analysis of basal-in mammary organoids in 384-well plates,” discussed above). Incucyte S3 (Satorious) microscope system was used for more frequently live-imaging for Endothelial organoids forming. The Incucyte S3 is capable of autofocusing on and individually imaging each well in the 384-well ULA plate. Endothelial organoid time course images were provided form Incucyte every hour, shown in FIG. 3. In FIG. 3, it can be seen that Endothelial organoid started aggregation just after few hours and formed with high circularity within few hours. The observed time to formation for the endothelial organoid aggregation is faster than epithelial cell formation.

Image analysis was performed using Python software that script was edited based on previous published work. (See, e.g., Lee et al. “High-throughput formation and image-based analysis of basal-in mammary organoids in 384-well plates,” discussed above). 384 well 4× bright field images from EVOS microscope were used to assessing cross-sectional area and circularity of organoids. As shown in FIGS. 4-6, included 40 data points of each culture days. All the data points also analyzed on this analysis plot based on the last culture day on 67, only have 40 organoids left. More than 100 sample points were used to analysis. From the day 1 to 67, cross-sectional area is about 0.55±0.02 mm² and circularities are from about 0.6 to about 0.85.

### Angiogenesis in Endothelial Organoid

While culturing endothelial organoids, it was confirmed that angiogenesis occurs within the organoid model. FIGS. 1 and 2 schematically represents how the organoids are formed. After centrifugation, the cells tend to cluster together, and within a few hours, they take on a 3D spherical shape characteristic of organoids. At this point, angiogenesis begins from the tip cell of the endothelial cell, and as the culture period continues, the vascular structures inside the organoid increase, resulting in a complex shape.

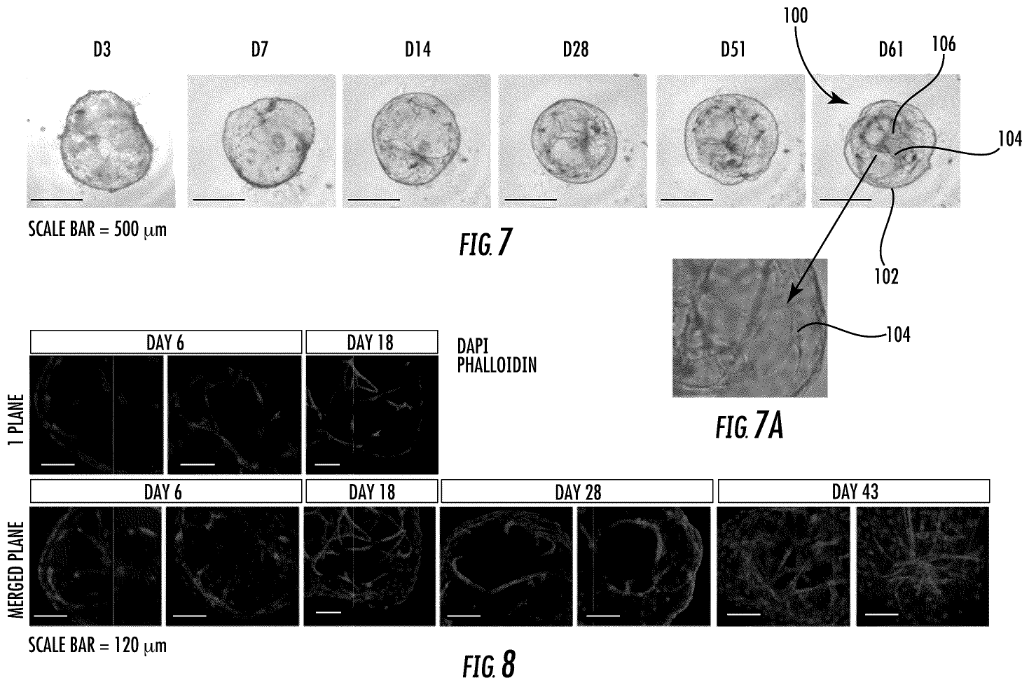

FIG. 7 shows the bright field (BF) images of organoids from day 3 to day 61 of culture. Although faint on days 3 and 7, thin line-like structures begin to appear. As the culture continues, the internal structure becomes more complex, which can be confirmed even on day 61 as the internal structure continues to develop. To understand the characteristics of this structure, we performed fluorescent staining and RNA Bulk sequencing. Complex signals from Integrin α6 were detected not only on the surface of the organoid but also internally, according to the structure in BF. The reason for the high noise is because a permeabilization step was not performed.

Also, the signal of actin was observed, a marker of the cell's cytoskeleton, from day 6 to day 3 of culture. As the culture period increased, the actin signal was continuously found, and its structure also appeared in a complex structure similar to what was found in the BF image. When the merged plane was observed rather than the single plane, it was confirmed that the inner structures inside the organoid are intricately connected to each other and also connected to the surface of the organoid. This is shown in FIG. 8, for example.

Furthermore, CD34 is known as a tip cell marker of endothelial cells, and it is known that angiogenesis, vasculogenesis, and sprouting begin from the tip cell. In most studies testing angiogenesis of endothelial cells, the signal of CD34 is analyzed and confirmed in various ways. This is described further in Hassanpour M, Salybekov A A, Kobayashi S and Asahara T (2023) “CD34 positive cells as endothelial progenitor cells in biology and medicine” Front. Cell Dev. Biol.,” which is incorporated by reference herein in its entirety.

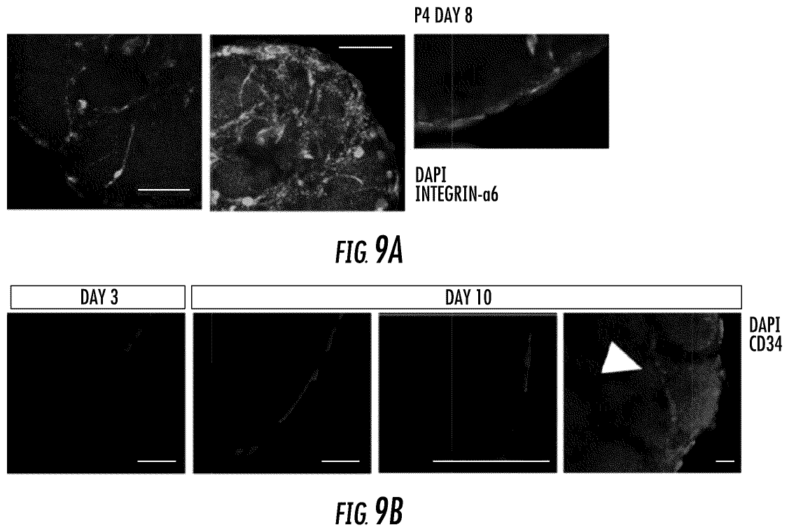

Looking at FIGS. 9a and 9b, the signal of CD34 was not well expressed on the 3rd day of culture, but on the 10th day of culture, it could be confirmed the fluorescence expressed along the surface of the organoid more clearly. Since the permeabilization step was not performed to check whether it is expressed on the surface during fluorescent staining, a lot of noise appeared when adjusting the contrast to check the inside. Nonetheless, it could still be confirmed that the fluorescence of CD34, a tip cell marker, was expressed along the sprouted structure inside the organoid.

### Cell Senescence Effect on Organoid Angiogenesis and Merging

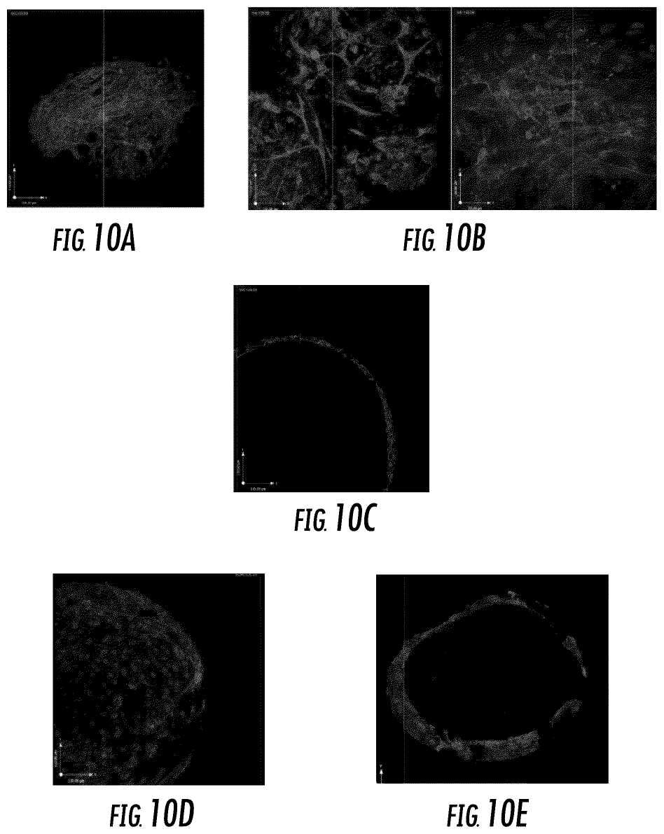

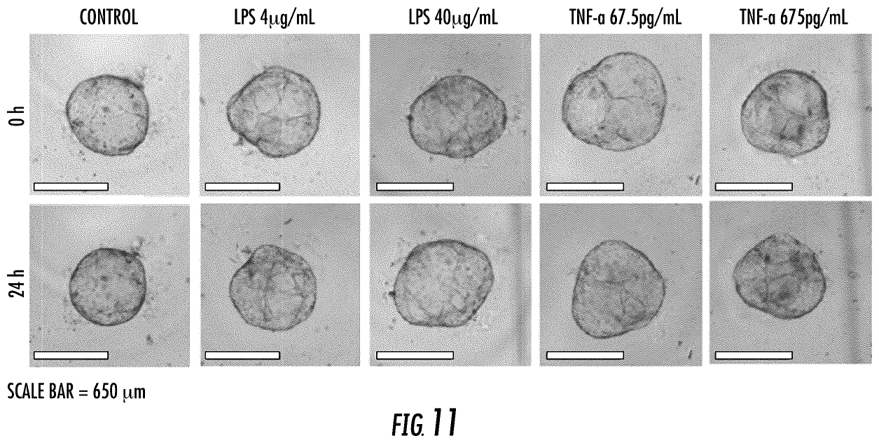

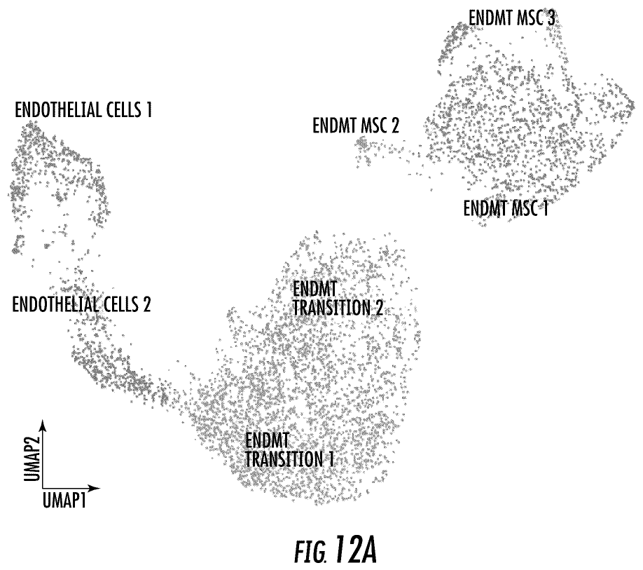

In the case of primary cells, they are extremely sensitive to the passage number, and various results accompany it. We have compared according to different passage numbers from P4 to a maximum of P16. It was confirmed that different results are shown in the shape and characteristics of the organoids. No matter how long the culture period is at high passage, it can be confirmed that no structure is formed inside the organoid. To confirm this, as shown in FIGS. 10a to 10e, actin fluorescence staining was performed, and despite being 24 days and 10 days of culture in P7 and 10, respectively, it was confirmed that no structure was formed inside the organoid. This is completely different from the structure and aspect appearing inside the P4 organoid as shown in FIG. 8. To analyze this more biologically, we compared P4 and P16 2D/3D sample sequencing data from bulk RNA sequencing.

Also, an organoid merging experiment was conducted to determine whether there are changes in the internal structure when organoids merge, and if so, whether it is influenced by the passage number. Organoids from P4 and P11 were cultured separately, and on the 7th day of culture, they were transferred to a 96-well plate, with 1-2 organoids placed together in each well, and maintained within an IncuCyte incubator.

### LPS and TNF-A Stimulated Endothelial Organoid to Expressing E-Selectin Signal

There is currently a lot of research being conducted on endothelial cells in relation to inflammation. Endothelial organoids are simulated with LPS and TNF-α, which are known to induce inflammation in cells, to check if they could also be applied. After 24 hours of treatment, no significant changes were observed in the bright field (BF). To investigate further, we performed fluorescent staining to detect the signal of E-selectin, a marker of inflammation. As can be seen in FIG. 11, compared to the control group, the organoids treated with LPS and TNF-α showed expression of the E-selectin signal on the surface of the organoids. Both LPS and TNF-α showed an increase in fluorescence proportional to their concentrations.

As shown and described herein, a 3D organoid model using only endothelial cells (ECs) derived from Human Umbilical Vein Endothelial Cells (HUVECs) was successfully cultivated and formed. Unlike previous studies that primarily used co-culture with other cells or models embedded in spheroids and Extracellular Matrix (ECM), the approach taken herein solely utilized ECs. A basic image analysis of the formed EC organoids was conducted, showing the ECs rapidly took on a spherical organoid shape within 1 to 3 hours and were cultivated for approximately 64 days. A new script for image analysis was developed by converting an already developed MATLAB code into Python. Using this code, it was observed that the cross-sectional area and circularity of the EC organoids remained largely unchanged from day 1 to day 67.

Furthermore, between approximately 3 to 7 days, tube-like structures similar to blood vessels within the organoids were visualized in Bright Field (BF) images (e.g., FIG. 7). Through actin fluorescence staining, the occurrence of vasculogenesis within the organoids was confirmed (e.g., as shown in FIG. 8). The fluorescence signal of CD34, a known tip cell marker indicative of sprouting, was also detected not only on the surface of the organoids but also in the sprouted structures within the organoids on day 10 of cultivation (e.g., as shown in FIGS. 9a and 9b).

Based on these findings, RNA bulk sequencing was conducted according to passage number and confirmed that the overall gene distribution of HUVECs at a lower passage was different from that at a higher passage. This difference was particularly noticeable in the aspect of vasculogenesis. This is consistent with the known sensitivity of ECs to the maturity of the cell, and it was confirmed that there are differences in the distribution of genes and the occurrence of vasculogenesis accordingly.

Finally, to examine the response of EC organoids under inflammatory conditions, the EC organoids were treated with Lipopolysaccharides (LPS) and Tumor Necrosis Factor-alpha (TNF-α). Following treatment, the expression of E-selectin, a marker of inflammation, on the surface of the organoids was confirmed through fluorescence signals.

The results observed and disclosed in study is significant as it is the first to form organoids using only endothelial cells. The novel and non-obvious in vitro cell construct disclosed herein presents a new cell model that can be applied in various fields, including cell function, external conditions like hypoxia and normoxia, and inflammation. This model, which allows for the examination of the effects of ECs alone, will be beneficial for research into disease models like cardiovascular disease (CVD), cancer, neurodegenerative disease, especially diseases known to accompany loss of pericytes, various drug treatments, and studies related to cell maturity. The results of this comprehensive study provides a novel perspective on the potential of EC organoids in vascular biology research.

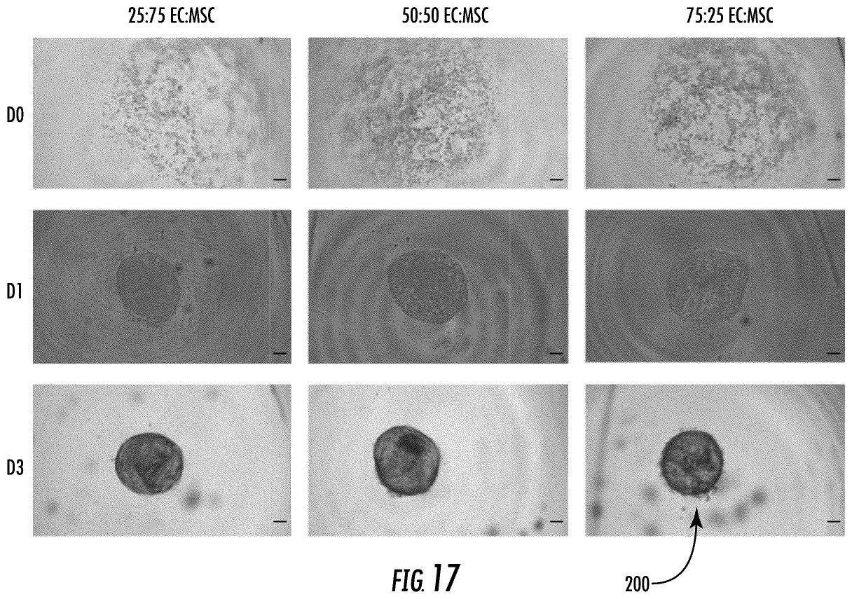

Referring now to FIGS. 16-21, Endothelial and Mesenchymal Co-Culture Organoids will be discussed. FIGS. 16-21 show results visualizing VMOs formed at different EC:MSC ratios allow long-term culturing and preserved morphology. FIG. 16 shows a schematic of the Vascular Mesenchymal Organoids (VMO) formation process, combining mesenchymal stem/stromal cells (MSCs), human umbilical vein endothelial cells (HUVECs), and Matrigel at 25:75, 50:50 and 75:25 EC:MSC ratios to generate organoids. FIG. 17 shows time-lapse images showing organoid formation during the first 3 days across different EC:MSC ratio, where organoids are fully formed by day 3 in all tested ratios.

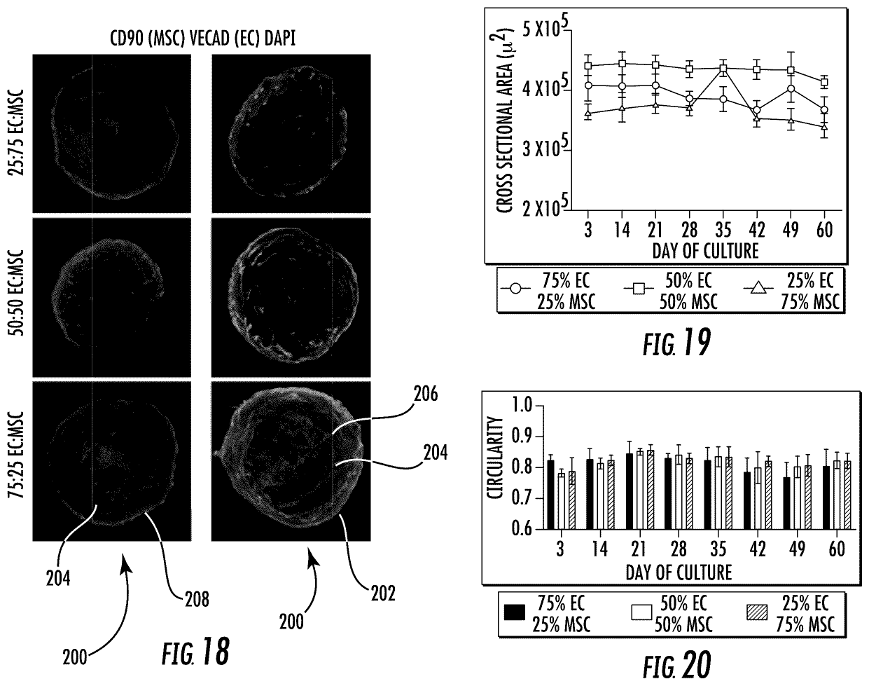

FIG. 18 shows D14 VMOs immunofluorescence analysis of organoids at different seeding ratios, demonstrating the presence of both VE-Cadherin+ endothelial cells (EC) and CD90+ MSCs. FIG. 19 shows a quantification of the cross-sectional area of organoids at different seeding ratios, showing that the 50:50 ratio produces the largest organoid area. FIG. 20 shows circularity measurements of organoids over 60 days of culture, showing stable (>0.8) circularity across all ratios.

FIG. 21 generally shows results visualizing VMOs present enhanced proliferation, improved vascularization and ECM assembly, and inflammation compared to spheroids. FIG. 21 specifically shows a comparison between D14 VMO and spheroid architecture, showing a mesh-like vasculature in the VMO and elongated vasculature in the spheroid. Here, spheroid vessels range from 4 to 8 μm while VMO vessel diameter extends up to 20 μm.

### Materials and Methods—Endothelial and Mesenchymal Co-Culture Organoids

Endothelial Cell Culture and Mesenchymal Stem Cell Culture (2D) Human umbilical vein endothelial cells (HUVECs) (ATCC, PCS-100-013) and bone marrow-derived mesenchymal stem cells (MSCs) (Rooster Bio, MSC 003) were cultured under standard conditions. HUVECs were maintained in Vascular cell basal medium (VCBM) supplemented with rh VEGF (5 ng/mL), rh EGF (5 ng/mL), rh FGF basic (5 ng/mL), rh IGF-1 (15 ng/mL), L-glutamine (10 mM), Heparin sulfate (0.75 Units/mL), Hydrocortisone (1 μg/mL), Ascorbic acid (50 μg/mL), 2% (vol/vol) fetal bovine serum (FBS) (ATCC, PCS-100-041), and 1% (vol/vol) pen/strep, while MSCs were cultured in Rooster-Nourish basal media (Rooster Bio, M22520, SU-003). The medium for both cell types was replaced every 3 days, and they were maintained in a humidified CO2 incubator at 37° C. with 5% CO2. Both HUVECs and MSCs cells were cultured in T75, T150, and T225 flasks and passaged between 80% to 90% confluency, with cells utilized up to passage number 4.

### Endothelial-MSC Organoid Culture (3D)

HUVEC Endothelial and MSC cells were added at different ratios (75:25, 50:50 and 75:25) to seeding media composed of 1.5% Matrigel (Corning #356-231, 10.7 mg/mL), 10% FBS (GeminiBio #900-108) and 0.24% methylcellulose (Methocel, Sigma Aldrich #94378) in growth media. The latter was formed by VCBM and DMEM-F12 (Gibco, #11320033) at 1:1 ratio and supplemented with EGF 5 ng/ml, L-glutamine 10 mM, VEGF 5 ng/ml, FGF 5 ng/ml, Heparin sulfate 0.75 Units/mL, Hydrocortisone hemisuccinate 1 μg/mL, and ascorbic acid 50 μg/mL. The final cell concentration was adjusted to 120 cells/μl. Organoid formation was conducted in 384-well U-bottom ULA plates (S-bio #MS-9384UZ, lot 20839306) with each well receiving a 25-μL drop followed by centrifugation at 1,000 rpm for 5 min (142 rcf, Thermo Scientific Sorvall ST16 centrifuge), resulting in organoids containing approximately 3,000 total cells. On day 3 of organoid culture, the media was exchanged three times to remove seeding supplements, utilizing a CyBio FeliX liquid handling machine (Analytik Jena). Media exchange occurred twice every 2-3 days.

### Endothelial-Osteo MSC Organoid Culture (3D)

Similar to Endothelial-MSC Organoid, HUVEC Endo and MSC cells were added at 1:1 ratio to seeding media composed of 1.5% Matrigel, 10% FBS and 0.24% methylcellulose in growth media. The latter was the previously mentioned media supplemented by 10 nM Dexamethasone (Sigma, D4902), 10 mM of glycerol 2-phosphate (Cayman Chemicals, #34516) and 200 μM of ascorbic acid (Fisher, A61-25). Organoid formation was conducted in 384-well U-bottom ULA plates with each well receiving a 25-μL drop followed by centrifugation at 1,000 rpm for 5 min and resulting in organoids containing approximately 3,000 total cells. Media was exchanged every 2-3 days utilizing a CyBio FeliX liquid handling machine from day 3 of culture.

### Cell Proliferation Assay

To assess cell proliferation in situ, the CellTiter-Glo® 3D Cell Viability Assay (Promega, G9681) was employed according to the manufacturer's instructions. Briefly, organoids were extracted from 384 ULA culture plate and transferred to a 96 well-plate, 50 μL of CellTiter-Glo® 3D reagent was added to each well containing the organoids. The plate was gently shaken for 5 minutes to induce cell lysis and ensure homogeneous distribution of the reagent. Following an additional 25-minute incubation period at room temperature to stabilize the luminescent signal, luminescence was measured using a microplate reader. Luminescence intensity was proportional to the number of viable cells in the organoids, allowing for quantitative assessment of cell proliferation.

### Whole Mount Immunofluorescence Staining and Imaging

Following organoid culture, whole mount immunofluorescence staining was conducted to visualize the expression of specific markers. Organoids were first fixed in 4% paraformaldehyde (PFA) for 1 hour at room temperature. Subsequently, organoids were permeabilized and blocked in a solution containing 10% donkey serum and 0.1% Triton X-100 for 1 hour to prevent nonspecific binding of antibodies. For primary antibody incubation, the following antibodies were added to the blocking solution and incubated overnight at 4° C.: rabbit anti-CD90 (1:100), goat anti-VE-CADHERIN (1:50), mouse anti-fibronectin (1:200), rabbit anti-laminin V (1:200), mouse anti-laminin V (1:200), rabbit anti-NG2 (1:200), mouse anti-alpha smooth muscle actin (αSMA) (1:200), and mouse anti-CD34 (1:500), goat anti-OPN (1:50).

After washing to remove unbound primary antibodies, organoids were incubated with appropriate secondary antibodies conjugated to fluorophores overnight at 4° C. The secondary antibodies used included donkey anti-mouse Alexa Fluor 555, donkey anti-rabbit Alexa Fluor 647, donkey anti-goat Alexa Fluor 647, and donkey anti-rabbit Alexa Fluor 488.

To visualize cellular nuclei or actin cytoskeleton, organoids were counterstained with DAPI (1:1000) or phalloidin Alexa Fluor 647 (1:500), respectively, for 1 hour at room temperature. Following immunostaining, organoids were subjected to tissue clearing by dehydration in a series of ethanol solutions (50%, 70%, 80%, 96%, and twice in 100%). Subsequently, organoids were cleared in BABB clearing agent (benzyl alcohol:benzyl benzoate, 1:2) to render them optically transparent for imaging. Finally, organoids were mounted on glass bottom chambers (ibidi, #1.5H) and imaged using spinning disk confocal using z-stack at a depth of 150 μm. Imaging was performed using both 20× and 60× objectives. Image analysis software, ImageJ and Volocity, were used for quantification and visualization of the immunofluorescence signals.

### Osteogenesis Quantification in Endothelia-Osteo MSC Organoids

Endothelial-Osteo MSC organoids underwent staining with Alizarin Red to visualize mineralized matrix deposition indicative of osteogenic differentiation. Organoids were first fixed in 4% paraformaldehyde (PFA) for 1 hour at room temperature. Following fixation, the organoids were washed with phosphate-buffered saline (PBS) and incubated with Alizarin Red staining solution prepared from the Osteogenesis Quantitation Kit (Sigma, ECM815) for 5 minutes at room temperature. The stained organoids were washed three times with PBS for 10 minutes each to remove excess staining solution and reduce background staining. The Alizarin Red staining allowed for the visualization of calcium deposits within the organoids, indicative of osteogenic differentiation of the MSCs within the co-culture system. After staining and washing steps, the organoids were mounted on glass slides and imaged using a brightfield microscope.

Quantitative PCR (qPCR) Analysis for Osteogenesis

Quantitative PCR (qPCR) analysis was conducted to quantify the expression of osteogenic markers during different time points of culture (days 14, 28, and 60) in the Endothelial-Osteo MSC organoids. For qPCR analysis, 36 organoids were combined in groups of 4 replicated. The organoids were washed three times with PBS to remove any residual culture media and debris. Subsequently, RNA extraction was performed by vortexing and lysing the organoids in RLT buffer (Qiagen, #79216) containing 1.0% beta-mercaptoethanol (Sigma-Aldrich, M3148).

Total RNA was then purified using RNA extraction columns (Qiagen, #74004) following the manufacturer's instructions. The purified RNA was subjected to reverse transcription using the Superscript VILO Master Mix (Thermo Fisher, #11755050) to synthesize complementary DNA (cDNA). TaqMan Fast advanced master mix assay (Thermo Fisher, #4444556) targeting the osteogenic marker genes SPP1 (osteopontin) and RUNX2 (runt-related transcription factor 2) were utilized for gene expression analysis. Quantitative PCR was performed using a QuantStudio 3 machine (Applied Biosystems).

### Results and Discussion—Endothelial and Mesenchymal Co-Culture Organoids

The vasculature is essential for tissue function and pathology. Spheroid co-cultures of endothelial and mesenchymal stem/stromal cells (MSCs) offer consistent structures, but the vascular components are short-lived. iPSC-derived vascular organoids can be long-lived but often have heterogeneous maturation and low reproducibility.

Embodiments of the in vitro cell construct as provided in this disclosure include consistently formed, free-floating, long-term Vascularized Mesenchymal Organoids (VMOs), formed by co-culturing human umbilical vein endothelial cells (HUVECs) and MSCs in a pre-gelled minimal Matrigel scaffold. VMOs support stable vasculature for up to 60 days, exhibiting dynamic tissue maturation, inflammation, extracellular matrix remodeling, and endothelial development. Compared to traditional spheroids, VMOs showed enhanced vascular complexity, sustained extracellular matrix production, and higher cellular proliferation. The system preserved MSC heterogeneity and included perivascular cell types, offering enhanced physiological relevance. Engraftment of breast cancer cells revealed stromal-tumor niches, enabling modeling of bone marrow metastasis and chemoprotection. This robust platform offers an alternative model for studying vascular biology, stromal dynamics, and cancer progression, with potential applications in drug testing.

As discussed in the previous section, endothelial cell only minimal Matrigel-based organoid models that provide consistently-shaped, free-floating structures with unconventional cellular organization compared to traditional matrix embedded systems were developed. In this section, an embodiment combining endothelial cells (EC) and MSCs is discussed. This section describes one or more embodiments of the EC MSC engineered Vascularized Mesenchymal Organoids (VMO), a platform capable of developing and maintaining long-term vasculature, preserving progenitor cells and incorporating supporting MSCs.

This platform was compared to established cell-aggregate spheroid systems, characterized the cell populations, and monitored suspended organoid evolution over 60 days. These results offer a robust, physiologically relevant, more proliferative, and long-term model for studying vasculature compared to space-filled spheroids while retaining the convenience of being consistently-shaped and readily accessed by cells and drugs by not being ECM gel embedded the way many organoids are cultured. As one potential application we also show promise of the VMO for modeling breast cancer metastasis to the bone marrow.

In the pursuit of a convenient and physiological vasculature, spheroid models have been widely used to co-culture ECs and MSCs. Co-culturing these cell types has been shown to improve angiogenesis and proliferation, as both populations produce soluble factors that promote mutual proliferation and angiogenesis. Furthermore, MSCs cultured in spheroids produce more soluble factors, including angiogenic factors, compared to their 2D counterparts 43-45. Despite these advantages, using conventional methods, spheroids struggle to develop a fully formed vasculature, often remaining in a pre-vascular state, which limits their use more to transplantation, where they can display functional vasculature in vivo.

The incorporation of hydrogels into conventional methods has improved vascularization in spheroids, leading to the formation of spindle-shaped vessels. However, these vessels fail to form a complete network, and typical culture times are limited to 21 days. Since the minimal Matrigel-based organoid technique has previously been used to combine different cell populations, embodiments described herein counterintuitively apply this technique to endothelial cells, mesenchymal stem cell co-cultures, with surprising and unexpected results.

Embodiments of the EC-MSC organoids described herein demonstrated both customization and robustness, as it can be formed with different EC:MSC ratios, with both populations coexisting without one overtaking the system, confirming the VMO multicellular compatibility. The VMOs maintained a homogeneous and stable architecture for 60 days, while MSC spheroids have shown stability for up to 30 days, which is significantly more than the conventional 20 or less days.

Moreover, the vasculature exhibited sensitivity to external agents, making the VMO platform promising for studying vascular pathologies such as acute or chronic inflammation and endothelial dysfunction. Serial 2D passaging of MSC cultures limit their differentiation capacity. MSCs cultured in VMOs appear to preserve their multipotency and were able to differentiate into mature osteoblasts. MSCs exhibit enhanced osteogenesis and multipotency when cultured in spheroids, and this process is further improved when co-cultured with ECs in agreement with our results.

Notably, embodiments of the VMOs cultured in regular media exhibited increased RUNX2 expression on Day 28, and enrichment of osteoblast differentiation pathways during the transition from Day 14 to Day 28. Embodiments of the novel EC-MSC 3D co-cultures have demonstrated natural osteogenesis driven by media composition and paracrine factors secreted in the co-culture. These findings are consistent with our observations of intrinsic osteogenesis in VMOs. The architecture displayed in VMOs shows MSCs both inside and outside, while ECs are located inside, forming the vasculature.

Cell localization is influenced by growth factors in other organoid models, where high concentrations tend to keep endothelial cells at the borders, while the absence of growth factors allows ECs to penetrate and form EC islets inside. Embodiments of the novel EC-MSC 3D co-culture utilizes four times fewer growth factors compared to the conditions that elicited that behavior, which could explain ECs localizing inside and forming a vasculature rather than islets.

The VMO model described herein exhibits a complex mesh-like vascular network that extends around the Matrigel core and demonstrates a more extensive vascularized area than spheroids, despite being seeded with the same number of ECs and MSCs. Moreover, it displays greater vascular complexity. While vessel diameters in spheroids remain below 10 microns, corresponding to capillaries, VMO vessels can reach up to 20 microns, mimicking the size of venules or small arterioles. This enhanced vascular network development could be attributed to the Matrigel, as previous studies have reported its role in modifying tubule structures in both EC-MSC and EC-only cultures.

Furthermore, hydrogel composition and stiffness play crucial roles in controlling vasculogenesis and network formation. We further characterized our ECs, which show a mixed gene expression of arterial and sinusoidal ECs, with an inclination toward sinusoidal ECs. Although HUVECS are not typically sinusoidal, in our system, they shift towards this phenotype. We note that the 60 day stability of vascular structures is rare not only compared to spheroids but in comparison with microchannel-based vascular systems as well.

Embodiments of the VMOs showed enrichment in several pathways involved in angiogenesis, vasculature and endothelium development when compared to spheroids, suggesting that VMOs are more conducive to angiogenesis. We also observed upregulation of RhoA GTPase pathways, which are associated with EC migration, tubule stress, and indirectly, angiogenesis through VEGF secretion suggesting a mechanosensing aspect in the vasculature development in VMOs. Enrichment of Notch-related pathways suggested that Notch signaling, likely promoted by MSCs, plays a role in endothelial development and sprouting, further supported by Notch downregulation observed simultaneously with D60 endothelium development decline.

A plausible explanation for the enhanced angiogenesis in VMOs could be the Matrigel scaffolding. Embodiments of the 3D co-cultures with scaffolds tended to exhibit improved angiogenesis, as they produce more soluble factors and demonstrate better spatial organization. This is particularly important because cell proximity influences angiogenesis and vascularization in 3D cultures, further supporting the role of the Matrigel core scaffolding in facilitating angiogenesis.

Beyond comparisons with spheroids, embodiments of the VMOs described herein demonstrated long-term vasculature preservation, with CD34+ progenitors present throughout the culture period, indicating that the vasculature remains active. The results suggest that angiogenesis begins as soon as the organoid forms, as evidenced by the upregulation of endothelial development on day 3, which coincides with the activation of hypoxic pathways and a fraction of dead cells at the organoid core, probably due to lack of nutrients, oxygen or soluble factors. This observation correlates with D14 immunofluorescence, which revealed a more developed vasculature compared to D3. These findings suggest a peak in angiogenesis during the first two weeks, followed by a decrease as the vasculature matured over the last month of culture.

On day 14, we observed upregulation of the Hippo signaling pathway, which is linked to angiogenesis regulation and junction formation. This suggests that the Hippo pathway may be modulating angiogenesis in the organoids and contributing to vasculature maturation by regulating cell junctions and vascular integrity. Despite the reduction in endothelial development pathways and GTPases observed at D60, the vasculature remains stable, as shown by day 60 immunofluorescence, suggesting a terminal organoid maturation rather than endothelium regression. Notably, the suppression of endothelium development on D14 and D60 coincides with the downregulation of the Rap1 signaling pathway, further supporting the role of mechanical force in regulating vasculature development and stability in embodiments of the novel system described herein.

Cell-cell communication analysis showed that at D3, MSCs were the primary contributors to angiogenic signaling, consistent with previous studies demonstrating their role in tubule formation and early vascular development 54. By D14, although overall angiogenic activity had declined, ECs became more prominently engaged in angiogenic pathways, indicating a transition from MSC-driven initiation to EC-mediated maturation and maintenance of a stable vascular network.

Extracellular matrix (ECM) plays a critical role in the vascular microenvironment by providing structural support, stabilizing the vasculature, and promoting angiogenesis. In embodiments of the models shown and described herein, ECM molecules were secreted across all tested ratios, with VMOs demonstrating a superior capacity to produce and maintain autologous ECM compared o spheroids. A specialized MSC subpopulation responsible for ECM production and remodeling was identified. On days 14 and 28, pathways related to ECM organization, ECM interaction, and focal adhesion were significantly upregulated, marking the peak of ECM remodeling. This corresponds with tissue development pathways observed on day 28, suggesting ongoing system maturation during the first month.

Additionally, the Wnt pathway is upregulated on D14, which has been previously correlated with enhanced MSC proliferation and ECM formation, as shown in studies where Wnt overexpression promoted wound repair and ECM regeneration. In contrast, ECM-related pathways were downregulated on day 60, indicating terminal maturation.

One notable difference between VMOs and spheroids is the presence of an inflammatory microenvironment in VMOs, as indicated by the upregulation of E-selectin and IL2 signaling pathway. This upregulation suggests an inflammatory response, which contrasts with the anti-inflammatory properties typically displayed by MSC spheroids. In microchannel devices, ECs often exhibit an inflammatory phenotype due to shear wall stress and mechanical forces exerted on them. Similarly, while a stiff ECM can induce angiogenesis and proliferation, prolonged exposure to high stiff substrate has been linked to inflammation and vascular dysfunction.

In VMOs, the Matrigel core could initially cause mechanical stretch on the ECs, leading to an inflammatory response as soon as it gets formed. Upregulation of RhoA GTPase pathways in VMOs aligns with previous studies that reported similar upregulation in ECs subjected to cyclic stretch and resulting in inflammatory phenotype expression. As the Matrigel is gradually digested and sprouting occurs, this inflammation decreases as observed in D3 to D14 and D14 to D28 shifts. On D14, we can observe a decrease in TGFβ and cytokine related inflammation, while several chemotaxis-related pathways are decreased on D28 indicating lower inflammation as the system matures. However, inflammation tends to increase between D28 and D60.

Unlike ECs, it has been reported that MSCs cultured on soft ECM become more inflammatory. During D28 to D60 transition several ECM pathways are downregulated, combined with increased Matrigel degradation due to blood vessel sprouting, this could explain the observed shift towards inflammation as the ECM undergoes remodeling and degradation, resulting in a softer matrix that may contribute to the restoration of an inflammatory environment.

Alternatively, the increase in inflammation may also be driven by the accumulation of senescent cells and elevated oxidative stress. Senescent cells are known to adopt a senescence-associated secretory phenotype, characterized by the release of pro-inflammatory factors. Since senescence signatures were enriched at early time points, the progressive accumulation of these cells may contribute to the reactivation of inflammation in the later stages of culture.

Similarly, we observed upregulation of ROS-related pathway at Day 60. Long-term culture under normoxic conditions has been previously associated with ROS accumulation and DNA damage, both of which are known to sustain chronic inflammatory responses. Along with ECM changes, cellular stress responses might contribute to promote the resurgence of inflammation during long-term culture. Bone marrow is known for containing heterogeneous MSC populations, and we have identified several MSC subpopulations in our system.

The presence of proliferative and fibroblastic subpopulations has been previously reported and aligns with our findings. The Progenitors cluster exhibits markers from different MSC subpopulations, indicating it could represent a combination of smaller subtypes that are not distinct enough to be separated. Interestingly, two clusters appear to be intermediate between ECs and MSCs, suggesting the possibility of endothelial-to-mesenchymal transition (EndMT), a process where ECs change their phenotype to become MSC-like. This transition is driven by inflammation or stiffness, both present in the VMO system.

Pericytes and mural cells are important perivascular cells involved in vascular proliferation and stability. We found a subpopulation of myofibroblasts localized near the blood vessels. Myofibroblasts can become perivascular during fibrosis or wound healing, a process that can be triggered by MSCs in response to TGFβ77, a top gene in the Progenitors cluster and regulated by Wnt signaling. Interestingly, SFRP1 a Wnt regulator, is a top upregulated gene in myfibroblast cluster. Vascular smooth muscle cells (VSMCs) are mural cells that can transition into a myofibroblastic phenotype under fibrotic conditions, making it difficult to clearly distinguish between the two populations. A small subset of these cells expresses CNN1, which is more commonly associated with VSMCs, suggesting a mix of both myofibroblast and VSMC.

Furthermore, we have observed smooth muscle-related pathways enriched during RNA-seq analysis, further supporting the possibility of a mixed population. iPSC-derived organoids, known for developing a complex vasculature, show perivascular cells expressing α-SMA and PDGFRB 21, 22, indicating the presence of perivascular cells. Similarly, VMO vasculature exhibits α-SMA perivascular cells, and the Progenitors cluster contains a PDGFRB+ subpopulation, suggesting the presence of mural cells in our system as well.

The involvement of different cellular components in the bone marrow niche in bone metastasis is well-established. Investigating the alterations in this microenvironment during invasion and therapy has become an emerging area of research. In our experiments, we used embodiments of the novel VMOs to engraft MCF7 breast cancer cells. The cancer cells formed niches that were preserved in VMOs but not in spheroids. Preserving these niches is crucial, as they constitute a pro-tumorigenic and pre-metastatic environment, rich in factors that influence cancer progression.

As breast cancer invasion is typically modeled in 2D cultures, which may poorly replicate the three-dimensional niche interactions when compared to 3D systems, which more accurately constitute a spatial microenvironment providing more physiologically relevant insights. We observed lower Ki67 activation in VMOs compared to 2D cultures, which may be attributed to the 3D culture system. Previous studies have shown that MSC spheroids and HUVECS/MSCs spheroids exhibit progressively lower Ki67 expression over 7 days of culture.

Furthermore, MSC/breast cancer coculture in spheroids contain higher fraction of G0/G1 cells. Similarly, MSC spheroids cultured on soft hydrogels also reported increased quiescence, suggesting that the Matrigel used in VMOs may contribute to reduced proliferation. This reduced proliferation is particularly relevant to imitate in vivo physiology, where tumor cells are highly heterogeneous, with a large proportion of quiescent cells capable of surviving therapy and contributing to tumor recurrence 84. The differential cell cycle progression related to 3D culture, as happens in VMOs, is difficult to model in 2D systems, further highlighting the importance of 3D platforms for studying tumor behavior. The reduced Ki67 activation in our VMO model likely reflects this characteristic of tumor cell behavior, underscoring the more physiologically relevant environment provided by 3D culture systems.

VMOs exhibit more protection than spheroids, likely due to the preservation of niches. Cancer cells are known to hijack these niches to promote chemoresistance through various mechanisms involving MSCs and ECs. In contrast, spheroids lost niches, forming larger aggregates that resemble already established breast cancer spheroids, limiting their ability to mimic the complex tumor microenvironment, which include normal cells and some vasculature, and its protective properties. Moreover, the attachment of breast cancer cells to selected adhesion molecules, found in Matrigel, has been reported to induce drug resistance, suggesting our platform could be suitable to model and study chemoresistance mechanisms.

### Comparison

### Endothelial-Only Organoids Vs Endothelial Mesenchymal Co-Culture Organoids

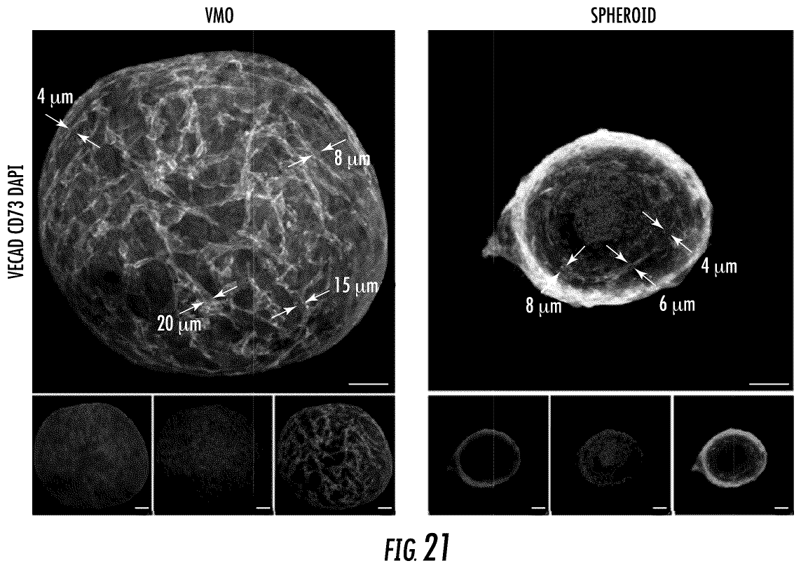

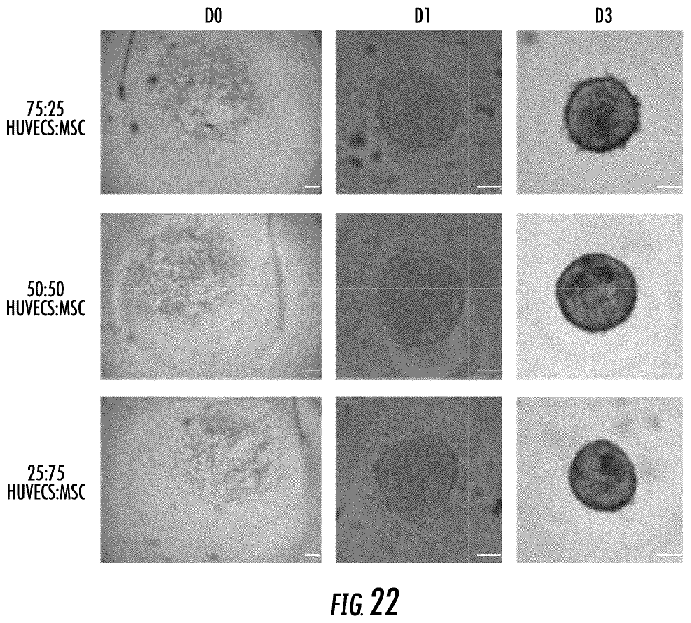

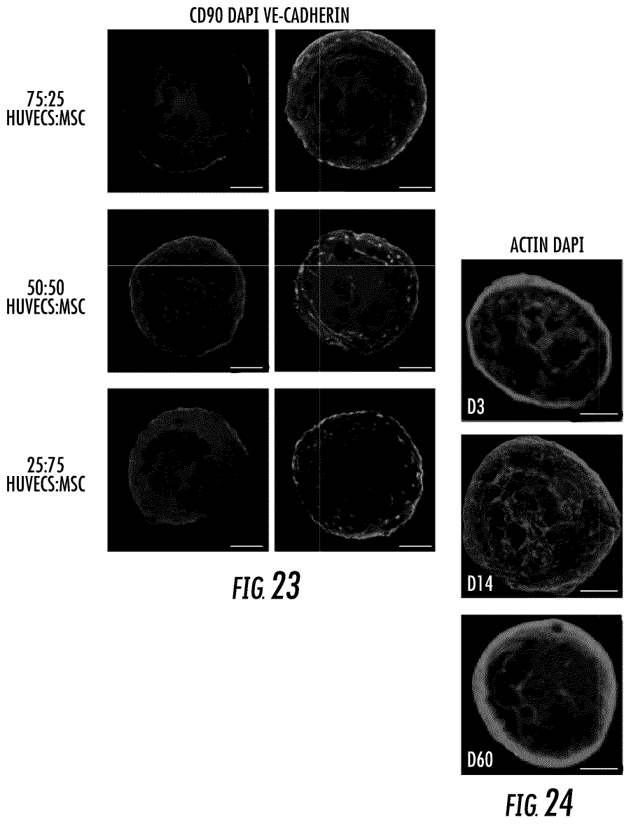

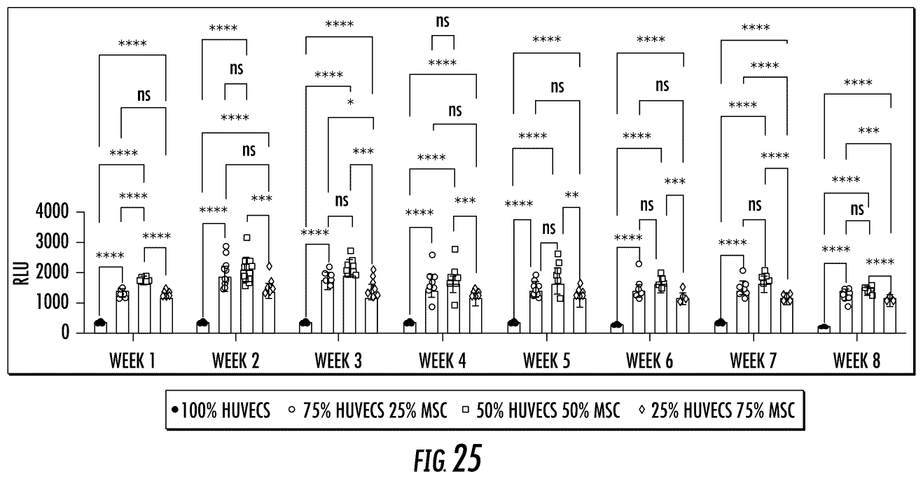

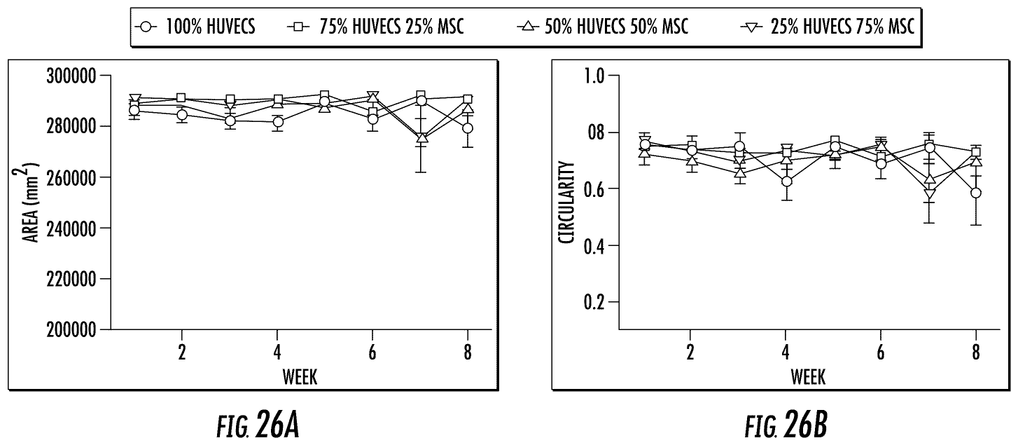

FIGS. 22-43 detail the comparison between Endothelial-Only Organoids vs Endothelial Mesenchymal Co-Culture Organoids, where different HUVEC:MSC ratios are studied. FIGS. 22-26b show organoid formation and growth kinetics. FIG. 22 shows organoids formed from different HUVEC:MSC ratios begin assembling within 24 hours and are fully formed after three days. This rapid development describes the effectiveness of the organoid formation method. in FIG. 23, MSC (CD90) and HUVEC (VE-CADHERIN) distribution within the organoids at different seeding ratios after 14 days of culture as shown. As is shown, varying the ratios of endothelial and mesenchymal cells show no impact on cellular organization. In FIG. 24, organoid core structure on culture days 3, 14, and 60 is shown. By day 60, the organoids have core cell growth circumventing the necrotic core characteristic of long-term cultured organoids. FIG. 25 shows organoid culture kinetics at different seeding ratios, where co-cultured organoids exhibit more cells throughout the culture period compared to HUVEC-only organoids. Organoids with a 50:50 HUVEC:MSC ratio display the highest cell number during the first week, while 75:25 and 50:50 ratios show the most proliferation throughout the 8-week culture period (n=15). Statistical significance: ****P<0.00005, ***P<0.0005, **P<0.005, *P<0.05, as determined by two-way ANOVA. FIG. 26a plots cross-sectional area of organoids indicating that organoids with different compositions exhibit a similar cross-sectional area throughout the study (n=20), FIG. 26b plots organoid circularity, where it appears to be maintained during long-term culture, with little change observed over time (n=20) suggesting a stable structural integrity during extended culture periods. Scale bars in FIGS. 22-24 represent 100 μm.

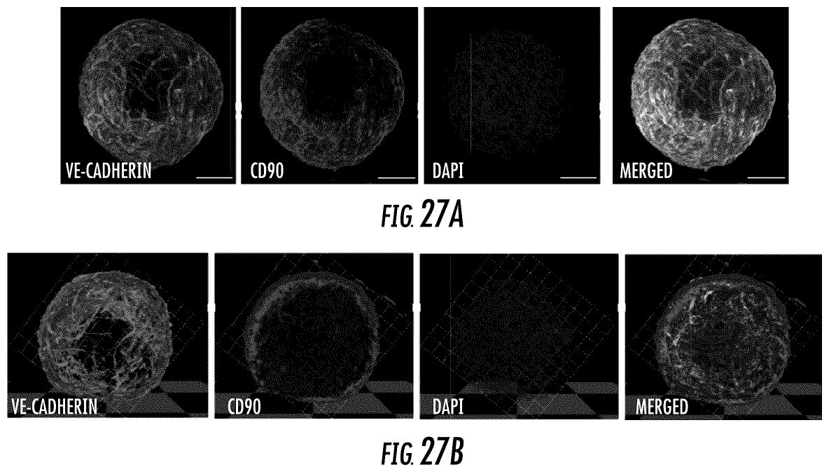

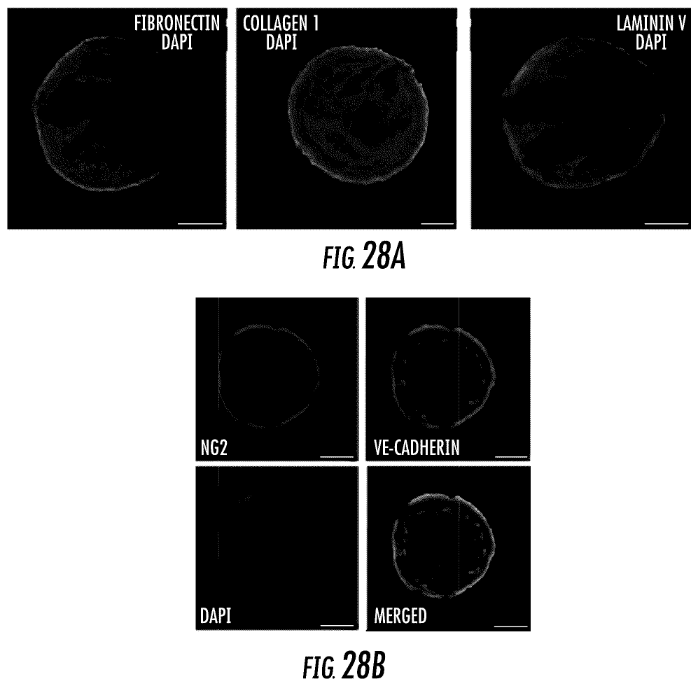

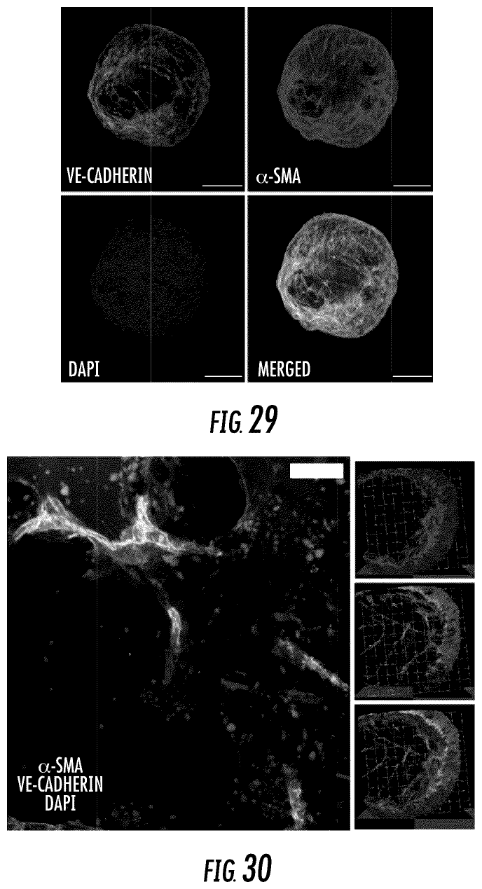

Referring now to FIGS. 27a-30, the organoid microenvironment is visualized. As can be seen in FIGS. 27a and 27b, the organoids exhibit an interconnected vasculature formed by HUVECs, which span from the outer surface and spread inward to create a complex network within the organoid. MSC can be observed on the organoid's surface, filling the spaces between the vascular structures. FIG. 27a shows a cross-section of the organoid, while 27b presents a 3D reconstruction. In FIG. 28a, it is shown that the system can produce extracellular matrix (ECM) molecules in situ, including fibronectin, collagen I, and laminin V, indicating active ECM production, deposition and autologous microenvironment formation within the organoids. Mural cells as shown in FIG. 28b, where NG2 mural cells are visible on the organoid's surface, suggesting the limited presence of pericytes or other supporting cells. FIGS. 29 and 30 show staining with alpha-SMA (smooth muscle actin), which reveals vascular smooth muscle cells surrounding the vasculature, forming a sheath-like structure. This feature suggests that the organoid has a more arterial or sinusoidal vasculature, rather than capillary. FIG. 29 shows the general distribution of vascular smooth muscle cells, while FIG. 30 provides a closer view, highlighting the denser arrangement. The scale bar represents 100 μm in FIGS. 27a-29, while in FIG. 30, the scale bar represents 20 μm.

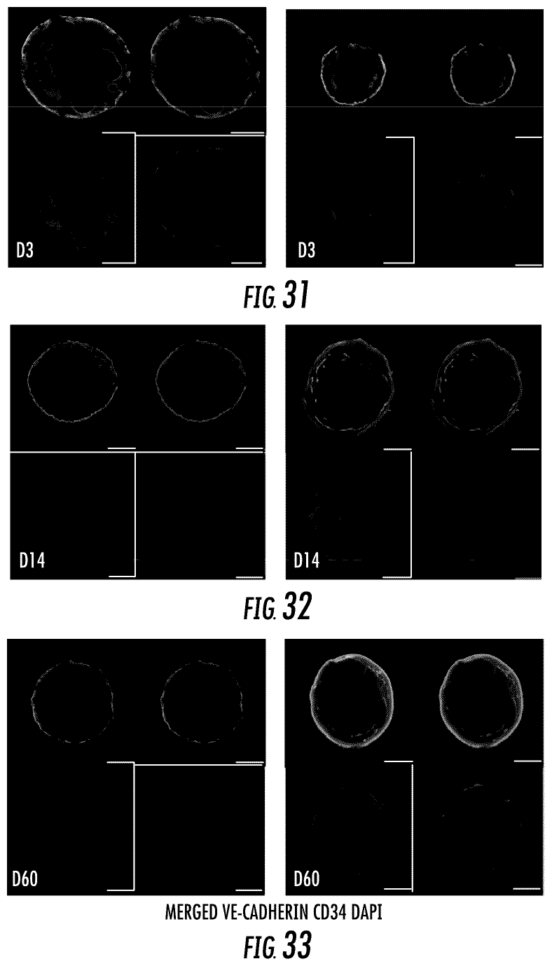

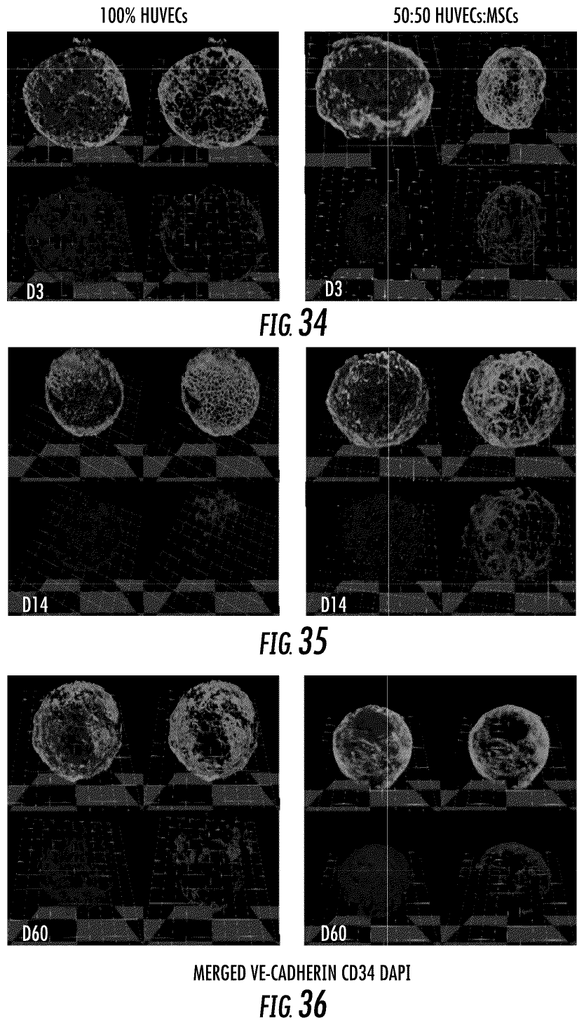

FIGS. 31-33 show vasculature formation in Co-Culture vs HUVEC Mono-Culture. Shown in FIG. 31, in recently formed organoids, HUVECs (VE-CADHERIN) and endothelial progenitors (CD34) are located along the organoid's edges in both co-culture and HUVEC mono-culture conditions. This observation suggests that initial vascularization begins at the outer surface in both scenarios. In FIG. 32, after 14 days of culture, the HUVEC mono-culture vasculature is observed primarily at the organoid's edges with sparse endothelial progenitors in the interior. In contrast, co-culture exhibits a more developed vasculature with endothelial progenitors growing along the blood vessels, indicating enhanced vascularization. As shown in FIG. 33, by day 60, in the mono-culture, endothelial cells grow in an array on the outer surface, with few progenitors observed within the internal structure. In contrast, the co-culture maintains a well-developed vasculature with supporting endothelial progenitors, suggesting a more stable and sustained vascular network. FIGS. 34-36 similarly show vasculature formation in Co-Culture vs HUVEC Mono-Culture like FIGS. 31-33, but FIGS. 34-36 are 3D reconstructions, where FIG. 34 is a 3D reconstruction of FIG. 31, FIG. 35 is a 3D reconstruction of FIG. 32, and FIG. 36 is a 3D reconstruction of FIG. 33. Scale bars in FIGS. 31-36 represent 100 μm.

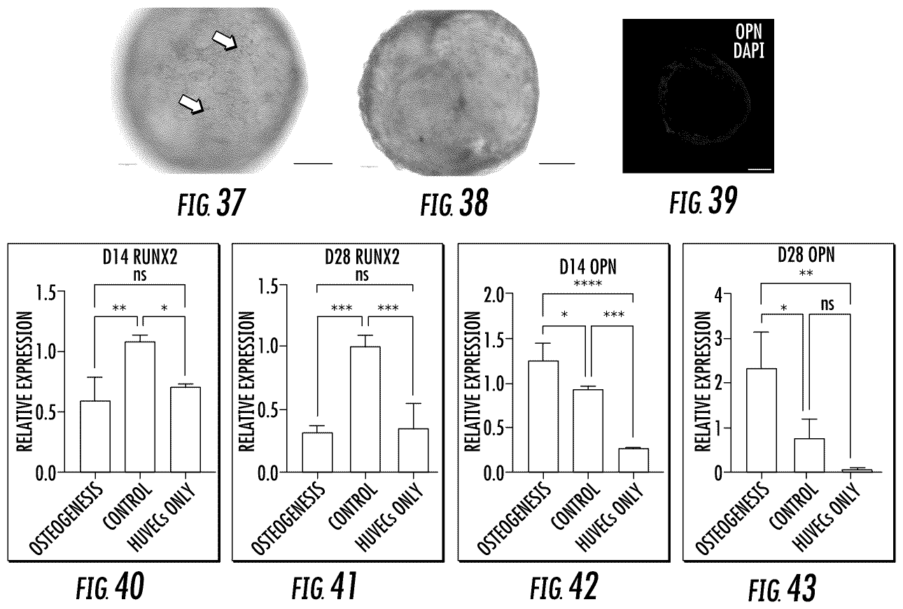

FIGS. 37 to 43 show data relating to osteogenesis in 50:50 Organoids cultured in osteogenic versus traditional growth media. FIGS. 37 and 38 are images showing organoids cultured for 3 weeks and stained with Alizarin Red to identify calcium nodules, a marker for bone formation. In FIG. 37, organoids cultured in osteogenic media show visible calcium nodules as red dots (highlighted by the arrow), indicating the initiation of bone formation. In FIG. 38, organoids cultured in traditional growth media, which serves as a control, do not exhibit any calcium nodules. FIG. 39 shows immunofluorescence on organoids subjected to osteogenesis for 4 weeks, which reveals the presence of osteopontin (OPN), indicating bone matrix formation. To measure the gene expression of RUNX2, a marker for immature osteoblasts, qPCR analysis was performed.

In FIG. 40, after 14 days, organoids in osteogenic media show lower expression of RUNX2, suggesting that limited number of immature osteoblasts. In contrast, organoids cultured in traditional growth media exhibit some osteo-committed cells. In FIG. 41, after 28 days, the organoids in osteogenic media maintain a lower level of RUNX2 expression, meanwhile, the control organoids in traditional media continue to show signs of early osteogenesis. FIG. 42 shows qPCR analysis of OPN gene expression, which indicates mature osteoblasts, demonstrates a marked increase after 14 days in organoids exposed to osteogenic media. This suggests a complete osteogenic differentiation compared to the controls, where the expression remains low. In FIG. 43, by day 28, the trend remains consistent, with organoids in osteogenic media exhibiting higher OPN expression levels than those in traditional media, indicating continued maturation of osteoblasts. Scale bars for FIGS. 37-39 represent 100 μm. Statistical analysis for FIGS. 40-43, ****P<0.00005, ***P<0.0005, **P<0.005, *P<0.05, as determined by single-way ANOVA.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

What is claimed is:

1. An in vitro cell construct, comprising:
a cellular layer including stretched endothelial cells having undergone a biological transformation defining a three-dimensional structure with vasculature formed around a basement membrane mimetic gel inner core.

2. The construct of claim 1, wherein the cellular layer is a monolayer comprising only endothelial cells and wherein the monolayer is the only cellular layer surrounding the inner core.

3. The construct of claim 1, wherein the inner core includes a hydrogel core formed of one or more of: Matrigel or Matrigel in combination with one or more of: collagens, fibronectin, vitronectin, hyaluronan, proteoglycan, glycoprotein, decellularized extracellular matrix, fibrin, fibrinogen, and/or DNA, RNA, and other nucleic acids.

4. The construct of claim 1, wherein the cellular layer is an exterior cellular layer surrounding the inner core, wherein the endothelial cells of the exterior cellular layer are configured to form the vasculature of the three-dimensional structure via invaginating from the exterior cellular layer into the inner core.

5. The construct of claim 1, wherein the biological transformation includes endothelial to mesenchymal cell transformation (EndMT) whereby the endothelial cells of the cellular layer transition to mesenchymal-type cells exhibiting structural properties of mesenchymal cells.

6. The construct of claim 5, wherein tension exerted on the stretched endothelial cells of the cellular layer induces the EndMT and generation of extracellular matrix, thereby stabilizing the three-dimensional structure.

7. The construct of claim 6, wherein the three-dimensional structure remains stable for more than 60 days.

8. The construct of claim 6, wherein the extracellular matrix is endothelium-supportive extracellular matrix configured to support the stability of three-dimensional structure for more than 70 days thereby providing culture conditions to allow the endothelial cells of the extracellular layer to undergo EndMT reversion.

9. The construct of claim 1, wherein the endothelial cells are derived from one or more of: human umbilical vein endothelial cells (HUVECs), cardiac endothelial cells, dermal endothelial cells, brain endothelial cells, cancer endothelial cells, retinal endothelial cells, arterial endothelial cell, placental endothelial cell, liver endothelial cell, kidney endothelial cell, bone marrow endothelial cells, liver sinusoidal endothelial cells, lymphatic endothelial cells, glomerular endothelial cells, placental endothelial cells, brain endothelial cells, retinal endothelial cells, cancer endothelial cells.

10. The construct of claim 1, wherein the cellular layer is a first cellular layer comprising endothelial cells, and further comprising a second cellular layer comprising mesenchymal or stromal cells co-cultured with the endothelial cells, wherein, based on a culture condition:
the first cellular layer and second cellular layer self-assemble into the three-dimensional structure around the core, such that the mesenchymal or stromal cells of the second cellular layer are outward of the core, and the endothelial cells of the first cellular layer are outward of mesenchymal or stromal cells of the second cellular layer; or
the endothelial cells remain within three-dimensional structure to form a vascular network but not form a core aggregate.

11. The construct of claim 10, wherein the mesenchymal or stromal cells are derived from one or more of: fibroblasts, smooth muscle cells, adipocytes and preadipocytes, pericytes, osteoblasts.

12. The construct of claim 1, further comprising one or more additional cell types incorporated into the cellular layer, the one or more additional cell types including: cancer cells, leukocytes, leukocyte progenitor or stem cells, and/or hematopoietic progenitor or stem cells, immune cells.

13. The construct of claim 1, wherein the construct is an organoid configured for use in diagnostics, cell production or engineering, pharmaceutical testing, and/or EndMT reversion study.

14. A method of producing an in vitro cell construct, comprising:
introducing a selection of endothelial cells to a seeding media having a basement membrane mimetic gel just sufficient in amount to for allowing the endothelial cells to encapsulate at least a portion of basement membrane mimetic as an exterior cellular layer, the seeding media further including on or more of fetal bovine serum and/or wound healing and/or cell migration-promoting growth factors to form a seeding mixture; and
centrifuging the seeding mixture to promote the endothelial cells to mechanically stretch around the portion of the basement membrane mimetic gel to form the exterior cellular layer thereby generating a three-dimensional structure where the exterior cellular layer is under tension.

15. The method of claim 14, wherein only endothelial are introduced to the seeding media, such that the only exterior cellular layer formed around the core includes only endothelial cells as a monolayer, and wherein the tension generated by the mechanical stretch of the endothelial cells induces a biological transformation of the endothelial cells.

16. The method of claim 15, wherein the biological transformation is EndMT.

17. The method of claim 14, further comprising, introducing a selection of mesenchymal or stromal cells to the seeding media such that the seeding mixture includes an endothelial cell and mesenchymal or stromal cell co-culture.

18. The method of claim 17, wherein the seeding mixture includes pro-angiogenic factors at a concentration configured to induce the endothelial cells to form a second exterior cellular layer outside of the mesenchymal cell exterior cellular layer.

19. The method of claim 17, wherein the seeding mixture does not include pro-angiogenic factors thereby causing the mesenchymal cells to remain within three-dimensional structure while the endothelial cells localize at an interior of the construct to form an aggregate at the core of the three-dimensional structure.

20. A method, comprising:
performing one or more of: diagnostics, cell production or engineering, pharmaceutical testing, and/or EndMT reversion study on the in vitro cell construct of claim 1.
